(19) United States
(12) Patent Application Publication
(10) Pub. No.: US 2012/0135995 A1
(43) Pub. Date: May 31, 2012

(54) FUNGICIDAL DIPHENYL-SUBSTITUTED PYRIDAZINES

(75) Inventor: Paula Louise Sharpe, Middleton, DE (US)

(73) Assignee: E.I. Dupont de Nemours and Company, Wilmington, DE (US)

(21) Appl. No.: 13/389,265

(22) PCT Filed: Aug. 2, 2010
(86) PCT No.: PCT/US10/44108

§ 371 Date: Feb. 7, 2012

Related U.S. Application Data
(60) Provisional application No. 61/232,121, filed on Aug. 7, 2009.

(51) Int. Cl. A01N 43/58; A01P 3/00; C07D 237/08

(52) U.S. Cl. 514/247; 544/224

(57) ABSTRACT

Disclosed are compounds of Formula 1, including all stereoisomers, N-oxides, and salts thereof,

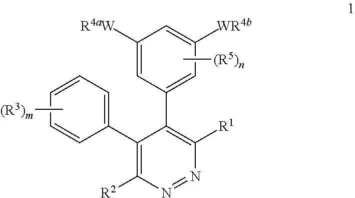

wherein
R¹, R², R³, R^{4a}, R^{4b}, R⁵, W, m and n are as defined in the disclosure.

Also disclosed are compositions containing the compounds of Formula 1 and methods for controlling plant disease caused by a fungal pathogen comprising applying an effective amount of a compound or a composition of the invention.

### FIELD OF THE INVENTION

This invention relates to certain pyridazines, their N-oxides, salts and compositions, and methods of their use as fungicides.

### BACKGROUND OF THE INVENTION

The control of plant diseases caused by fungal plant pathogens is extremely important in achieving high crop efficiency. Plant disease damage to ornamental, vegetable, field, cereal, and fruit crops can cause significant reduction in productivity and thereby result in increased costs to the consumer. Many products are commercially available for these purposes, but the need continues for new compounds which are more effective, less costly, less toxic, environmentally safer or have different sites of action.

PCT Patent Publications WO 2006/001175 and WO 2005/121104 disclose certain pyridazine derivatives of Formula i

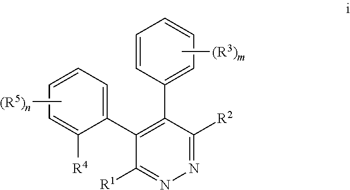

and their use as fungicides.

PCT Patent Publications WO 2008/049584 and WO 2008/049585 disclose certain pyridazine derivatives of Formula ii

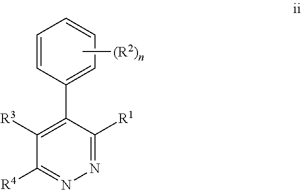

and their use as fungicides.

PCT Patent Publication WO 2008/089934 and German Patent Application DE 102008000872 A1 disclose certain pyridazine derivatives of Formula iii

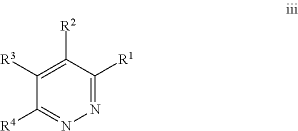

and their use as fungicides.

### SUMMARY OF THE INVENTION

This invention is directed to compounds of Formula 1 (including all stereoisomers), N-oxides, and salts thereof, agricultural compositions containing them and their use as fungicides:

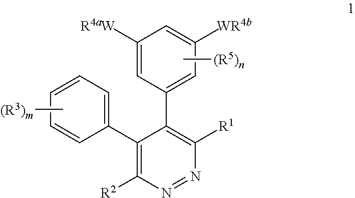

wherein
each W is independently O or S;
R¹ and R² are each independently H, halogen, cyano, C₁-C₆ alkyl, C₂-C₆ alkenyl, C₂-C₆ alkynyl, C₃-C₆ cycloalkyl, C₁-C₆ haloalkyl, C₂-C₆ haloalkenyl, C₁-C₆ hydroxyalkyl, C₂-C₆ cyanoalkyl, C₁-C₆ alkoxy, C₁-C₆ haloalkoxy, C₁-C₆ alkylthio, C₁-C₆ haloalkylthio, C₂-C₆ alkylcarbonyl or C₂-C₆ alkoxycarbonyl;
each R³ is independently halogen, cyano, nitro, C₁-C₆ alkyl, C₂-C₆ alkenyl, C₂-C₆ alkynyl, C₃-C₆ cycloalkyl, C₁-C₆ haloalkyl, C₂-C₆ haloalkenyl, C₁-C₆ alkoxy, C₁-C₆ haloalkoxy, C₁-C₆ alkylthio, C₁-C₆ haloalkylthio, C₂-C₆ alkylcarbonyl, C₂-C₆ alkoxycarbonyl, C₂-C₆ alkylaminocarbonyl, C₃-C₆ dialkylaminocarbonyl or C₃-C₆ trialkylsilyl;
R^{4a} and R^{4b} are each independently C₁-C₄ alkyl, C₁-C₄ haloalkyl or C₃-C₆ cycloalkyl;
each R⁵ is independently halogen, cyano, nitro, C₁-C₄ alkyl, C₁-C₄ haloalkyl, C₁-C₄ alkoxy, C₁-C₄ haloalkoxy, C₁-C₄ alkylthio or C₁-C₄ haloalkylthio;
m is 1, 2, 3, 4 or 5; and
n is 0, 1 or 2;

provided that:
(a) when R¹ is H, chloro, cyano or methoxy, then R² is not the same as R¹; and
(b) the compound is other than 4-(2,6-difluorophenyl)-5-(3,5-dimethoxyphenyl)-3-methyl-6-(1-methylethenyl)pyridazine, 4-(2,4-di fluorophenyl)-5-(3,5-dimethoxyphenyl)-3-methyl-6-(1-methylethenyl)pyridazine or 4-(3,5-dimethoxyphenyl)-5-(4-methoxyphenyl)-6-methyl-3-(1-methylethenyl)pyridazine.

More particularly, this invention pertains to a compound of Formula 1 (including all stereoisomers), an N-oxide, or a salt thereof.

This invention also relates to a fungicidal composition comprising (a) a compound of the invention (i.e. in a fungicidally effective amount); and (b) at least one additional component selected from the group consisting of surfactants, solid diluents and liquid diluents.

This invention also relates to a fungicidal composition comprising (a) a compound of the invention; and (b) at least one other fungicide (e.g., at least one other fungicide having a different site of action).

This invention further relates to a method for controlling plant diseases caused by fungal plant pathogens comprising applying to the plant or portion thereof, or to the plant seed, a fungicidally effective amount of a compound of the invention (e.g., as a composition described herein).

### DETAILS OF THE INVENTION

As used herein, the terms “comprises”, “comprising”, “includes”, “including”, “has”, “having”, “contains”, “containing”, “characterized by” or any other variation thereof, are intended to cover a non-exclusive inclusion, subject to any limitation explicitly indicated. For example, a composition, mixture, process or method that comprises a list of elements is not necessarily limited to only those elements but may include other elements not expressly listed or inherent to such composition, mixture, process or method.

The transitional phrase “consisting of” excludes any element, step, or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Where applicants have defined an invention or a portion thereof with an open-ended term such as “comprising,” it should be readily understood that (unless otherwise stated) the description should be interpreted to also describe such an invention using the terms “consisting essentially of” or “consisting of.”

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As referred to in the present disclosure and claims, “plant” includes members of Kingdom Plantae, particularly seed plants (Spermatopsida), at all life stages, including young plants (e.g., germinating seeds developing into seedlings) and mature, reproductive stages (e.g., plants producing flowers and seeds). Portions of plants include geotropic members typically growing beneath the surface of the growing medium (e.g., soil), such as roots, tubers, bulbs and corms, and also members growing above the growing medium, such as foliage (including stems and leaves), flowers, fruits and seeds.

As referred to herein, the term “seedling”, used either alone or in a combination of words means a young plant developing from the embryo of a seed.

In the above recitations, the term “alkyl”, used either alone or in compound words such as “alkylthio” or “haloalkyl” includes straight-chain or branched alkyl such as methyl, ethyl, n-propyl, i-propyl, or the different butyl, pentyl or hexyl isomers. “Alkenyl” includes straight-chain or branched alkenes such as ethenyl, 1-propenyl, 2-propenyl, and the different butenyl, pentenyl and hexenyl isomers. “Alkenyl” also includes polyenes such as 1,2-propadienyl and 2,4-hexadienyl. “Alkynyl” includes straight-chain or branched alkynes such as ethynyl, 1-propynyl, 2-propynyl and the different butynyl, pentynyl and hexynyl isomers. “Alkynyl” can also include moieties comprised of multiple triple bonds such as 2,5-hexadiynyl. The term “cycloalkyl” denotes a saturated carbocyclic ring consisting of from 3 to 6 carbon atoms linked to one another by single bonds. Examples of “cycloalkyl” include cyclopropyl, cyclobutyl, cyclopentyl and cyclohexyl.

“Alkoxy” includes, for example, methoxy, ethoxy, n-propyloxy, i-propyloxy and the different butoxy, pentoxy and hexyloxy isomers. “Alkylthio” includes branched or straight-chain alkylthio moieties such as methylthio, ethylthio, and the different propylthio, butylthio, pentylthio and hexylthio isomers.

“Hydroxyalkyl” denotes an alkyl group substituted with one hydroxy group. Examples of “hydroxyalkyl” include HOCH₂CH₂, CH₃CH₂(OH)CH and HOCH₂CH₂CH₂CH₂. “Cyanoalkyl” denotes an alkyl group substituted with one cyano group. Examples of “cyanoalkyl” include NCCH₂, NCCH₂CH₂ and CH₃CH(CN)CH₂.

“Trialkylsilyl” includes 3 branched and/or straight-chain alkyl radicals attached to and linked through a silicon atom, such as trimethylsilyl, triethylsilyl and tert-butyldimethylsilyl.

“Alkylcarbonyl” denotes a straight-chain or branched alkyl group bonded to a C(═O) moiety. Examples of “alkylcarbonyl” include CH₃C(═O), CH₃CH₂CH₂C(═O) and (CH₃)₂CHC(═O). Examples of “alkoxycarbonyl” include CH₃C(═O), CH₃CH₂OC(═O), CH₃CH₂CH₂C(═O), (CH₃)₂CHOC(═O) and the different pentoxy- or hexoxycarbonyl isomers. Examples of “alkylaminocarbonyl” include CH₃NHC(═O), CH₃CH₂NHC(═O), CH₃CH₂CH₂NHC(═O), (CH₃)₂CHNHC(═O) and the different pentylamino- or hexylaminocarbonyl isomers. Examples of “dialkylaminocarbonyl” include (CH₃)₂NC(═O), (CH₃CH₂)₂NC(═O), CH₃CH₂(CH₃)NC(═O) and (CH₃)₂CH(CH₃)NC(═O).

The term “halogen”, either alone or in compound words such as “haloalkyl”, or when used in descriptions such as “alkyl substituted with halogen” includes fluorine, chlorine, bromine or iodine. Further, when used in compound words such as “haloalkyl”, or when used in descriptions such as “alkyl substituted with halogen” said alkyl may be partially or fully substituted with halogen atoms which may be the same or different. Examples of “haloalkyl” or “alkyl substituted with halogen” include F₃C, ClCH₂, CF₃CH₂ and CF₃CCl₂. The terms “haloalkoxy”, “haloalkenyl” and “haloalkylthio”, are defined analogously to the term “haloalkyl”. Examples of “haloalkoxy” include CF₃O, CCl₃CH₂O, F₂CHCH₂CH₂O and CF₃CH₂O. Examples of “haloalkylthio” include CCl₃S, CF₃S, CCl₃CH₂S and ClCH₂CH₂CH₂S. Examples of “haloalkenyl” include (Cl)₂C═CHCH₂ and CF₃CH₂CH═CHCH₂.

The total number of carbon atoms in a substituent group is indicated by the “Cᵢ-Cⱼ” prefix where i and j are numbers from 1 to 6. For example, C₁-C₄ alkylcarbonyl designates methylcarbonyl through butylcarbonyl; C₂ alkoxy designates CH₃CH₂O; C₃ alkoxy designates, for example, CH₃CH(CH₃)O, CH₃CH₂CH₂O or (CH₃)₂CHO; and C₄ alkoxy designates the various isomers of an alkoxy group containing a total of four carbon atoms, examples including CH₃CH₂CH₂CH₂O and (CH₃)₂CHCH₂O.

The term “unsubstituted” in connection with a group such as a ring means the group does not have any substituents other than its one or more attachments to the remainder of Formula 1. The term “optionally substituted” means that the number of substituents can be zero. Commonly, the number of optional substituents (when present) range from 1 to 3. As used herein, the term “optionally substituted” is used interchangeably with the phrase “substituted or unsubstituted” or with the term “(un)substituted.”

When a compound is substituted with a substituent bearing a subscript that indicates the number of said substituents can exceed 1, said substituents (when they exceed 1) are independently selected from the group of defined substituents, (e.g., (R³)ₘ wherein m is 1, 2, 3, 4 or 5). When a variable group is shown to be optionally attached to a position, for example (R⁵)ₙ wherein n may be 0, then hydrogen may be at the position even if not recited in the variable group definition. When one or more positions on a group are said to be “not substituted” or “unsubstituted”, then hydrogen atoms are attached to take up any free valency.

Compounds of this invention can exist as one or more stereoisomers. The various stereoisomers include enantiomers, diastereomers, atropisomers and geometric isomers. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers. The compounds of the invention may be present as a mixture of stereoisomers, individual stereoisomers or as an optically active form. Of particular note are atropisomers, which are stereoisomeric conformations of a molecule that occur when rotation about a single bond is restricted such that interconversion is slow enough to allow separation. Restricted rotation of one or more bonds is a result of steric interaction with other parts of the molecule. In the present invention, compounds of Formula 1 can exhibit atropisomerism when the energy barrier to free rotation around a single unsymmetrical bond (i.e. where substituents on the phenyl rings render the bond unsymmetrical) is sufficiently high that separation of isomers is possible. Atropisomerism is defined to exist where the isomers have a half-life of at least 1000 seconds, which is a free energy barrier of at least about 22.3 kcal mol⁻¹ at about 20° C. (Oki, Topics in Stereochemistry, Vol. 14, John Wiley & Sons, Inc., 1983). One skilled in the art will appreciate that one atropisomer may be more active and/or may exhibit beneficial effects when enriched relative to the other atropisomer or when separated from the other atropisomer. Additionally, the skilled artisan knows how to separate, enrich, and/or to selectively prepare said atropisomers. Further description of atropisomers can be found in March, Advanced Organic Chemistry, 101-102, 4^{th} Ed. 1992; Oki, Topics in Stereochemistry, Vol. 14, John Wiley & Sons, Inc., 1983 and Gawronski et al, Chirality 2002, 14, 689-702. This invention includes compounds or compositions that are enriched in an atropisomer of Formula 1 compared to other atropisomers of the compounds. Also included are the essentially pure atropisomers of compounds of Formula 1.

One skilled in the art will appreciate that not all nitrogen-containing heterocycles can form N-oxides since the nitrogen requires an available lone pair for oxidation to the oxide. One skilled in the art will also recognize that tertiary amines can form N-oxides. Synthetic methods for the preparation of N-oxides of heterocycles and tertiary amines are very well known by one skilled in the art including the oxidation of heterocycles and tertiary amines with peroxy acids such as peracetic and m-chloroperbenzoic acid (MCPBA), hydrogen peroxide, alkyl hydroperoxides such as t-butyl hydroperoxide, sodium perborate, and dioxiranes such as dimethyldioxirane. These methods for the preparation of N-oxides have been extensively described and reviewed in the literature, see for example: T. L. Gilchrist in Comprehensive Organic Synthesis, vol. 7, pp 748-750, S. V. Ley, Ed., Pergamon Press; M. Tisler and B. Stanovnik in Comprehensive Heterocyclic Chemistry, vol. 3, pp 18-20, A. J. Boulton and A. McKillop, Eds., Pergamon Press; M. R. Grimmett and B. R. T. Keene in Advances in Heterocyclic Chemistry, vol. 43, pp 149-161, A. R. Katritzky, Ed., Academic Press; M. Tisler and B. Stanovnik in Advances in Heterocyclic Chemistry, vol. 9, pp 285-291, A. R. Katritzky and A. J. Boulton, Eds., Academic Press; and G. W. H. Cheeseman and E. S. G. Werstiuk in Advances in Heterocyclic Chemistry, vol. 22, pp 390-392, A. R. Katritzky and A. J. Boulton, Eds., Academic Press.

One skilled in the art recognizes that because in the environment and under physiological conditions salts of chemical compounds are in equilibrium with their corresponding nonsalt forms, salts share the biological utility of the nonsalt forms. Thus a wide variety of salts of the compounds of Formula 1 are useful for control of plant diseases caused by fungal plant pathogens (i.e. are agriculturally suitable). The salts of the compounds of Formula 1 include acid-addition salts with inorganic or organic acids such as hydrobromic, hydrochloric, nitric, phosphoric, sulfuric, acetic, butyric, fumaric, lactic, maleic, malonic, oxalic, propionic, salicylic, tartaric, 4-toluenesulfonic or valeric acids. Accordingly, the present invention comprises compounds selected from Formula 1, N-oxides and agriculturally suitable salts thereof.

Compounds selected from Formula 1, stereoisomers, N-oxides, and salts thereof, typically exist in more than one form, and Formula 1 thus includes all crystalline and non-crystalline forms of the compounds that Formula 1 represents. Non-crystalline forms include embodiments which are solids such as waxes and gums as well as embodiments which are liquids such as solutions and melts. Crystalline forms include embodiments which represent essentially a single crystal type and embodiments which represent a mixture of polymorphs (i.e. different crystalline types). The term “polymorph” refers to a particular crystalline form of a chemical compound that can crystallize in different crystalline forms, these forms having different arrangements and/or conformations of the molecules in the crystal lattice. Although polymorphs can have the same chemical composition, they can also differ in composition due the presence or absence of co-crystallized water or other molecules, which can be weakly or strongly bound in the lattice. Polymorphs can differ in such chemical, physical and biological properties as crystal shape, density, hardness, color, chemical stability, melting point, hygroscopicity, suspensibility, dissolution rate and biological availability. One skilled in the art will appreciate that a polymorph of a compound represented by Formula 1 can exhibit beneficial effects (e.g., suitability for preparation of useful formulations, improved biological performance) relative to another polymorph or a mixture of polymorphs of the same compound represented by Formula 1. Preparation and isolation of a particular polymorph of a compound represented by Formula 1 can be achieved by methods known to those skilled in the art including, for example, crystallization using selected solvents and temperatures.

Embodiments of the present invention as described in the Summary of the Invention include those described below. In the following Embodiments, Formula 1 includes N-oxides and salts thereof, and reference to “a compound of Formula 1” includes the definitions of substituents specified in the Summary of the Invention unless further defined in the Embodiments.

Embodiment 1. A compound of Formula 1 wherein each W is O.

Embodiment 2. A compound of Formula 1 or Embodiment 1 wherein R¹ and R² are each independently H, halogen, C₁-C₄ alkyl, C₂-C₄ alkenyl, C₁-C₄ haloalkyl, C₁-C₄ alkoxy, C₂-C₄ alkylcarbonyl, C₁-C₄ hydroxyalkyl or C₂-C₄ cyanoalkyl.

Embodiment 2a. A compound of Embodiment 2 wherein R¹ and R² are each independently H, halogen, C₁-C₄ alkyl, C₂-C₄ alkenyl, C₁-C₄ haloalkyl, C₁-C₄ alkoxy, C₂-C₄ alkylcarbonyl or C₁-C₄ hydroxyalkyl.

Embodiment 3. A compound of Embodiment 2a wherein R¹ and R² are each independently H, halogen, C₁-C₂ alkyl, C₂ alkenyl, C₁-C₂ alkoxy, C₂ alkylcarbonyl or C₁-C₃ hydroxyalkyl.

Embodiment 4. A compound of Embodiment 3 wherein R¹ and R² are each independently H, Br, Cl, F, methyl, C₂ alkenyl or methoxy.

Embodiment 4a. A compound of Embodiment 4 wherein R¹ and R² are each independently H, Br, Cl, methyl, C₂ alkenyl or methoxy.

Embodiment 4b. A compound of Embodiment 4a wherein R¹ and R² are each independently H, Cl or methyl.

Embodiment 5. A compound of Embodiment 4 wherein R¹ and R² are each independently Cl, F or methyl.

Embodiment 7. A compound of Embodiment 5 wherein R¹ and R² are each independently Cl or methyl.

Embodiment 7. A compound of Embodiment 6 wherein R¹ and R² are each methyl.

Embodiment 8. A compound of Formula 1 or any one of Embodiments 1 through 7 wherein when R¹ and R² are each independently Cl or methyl, then one of R¹ and R² is Cl and the other one of R¹ and R² is methyl.

Embodiment 9. A compound of Formula 1 or any one of Embodiments 1 through 8 wherein each R³ is independently halogen, cyano, C₁-C₃ alkyl, C₁-C₃ haloalkyl, C₁-C₃ alkoxy, C₁-C₃ haloalkoxy or C₁-C₃ alkylthio.

Embodiment 10. A compound of Embodiment 9 wherein each R³ is independently Cl, F, cyano, methyl, methoxy or methylthio.

Embodiment 11. A compound of Embodiment 10 wherein each R³ is independently Cl, F, methyl or methoxy.

Embodiment 12. A compound of Embodiment 11 wherein each R³ is independently F or methoxy.

Embodiment 13. A compound of Embodiment 12 wherein each R³ is F.

Embodiment 14. A compound of Formula 1 or any one of Embodiments 1 through 13 wherein m is 2 or 3.

Embodiment 15. A compound of Embodiment 14 wherein m is 3.

Embodiment 16. A compound of Embodiment 14 wherein m is 2.

Embodiment 17. A compound of Formula 1 or any one of Embodiments 1 through 16 wherein at least one R³ substituent is attached at an ortho position.

Embodiment 18. A compound of Embodiment 17 wherein two R³ substituents are attached at the ortho positions.

Embodiment 19. A compound of Formula 1 or any one of Embodiments 1 through 16 wherein one R³ substituent is attached at an ortho position and one R³ substituent is attached at the para position.

Embodiment 20. A compound of Formula 1 or any one of Embodiments 1 through 15 wherein two R³ substituents are attached at the ortho positions and one R³ substituent is attached at a meta position or the para position.

Embodiment 20a. A compound of Embodiment 20 wherein two R³ substituents are attached at the ortho positions and one R³ substituent is attached at the para position.

Embodiment 21. A compound of Embodiment 20 wherein two R³ substituents are attached at the ortho positions and one R³ substituent is attached at a meta position.

Embodiment 22. A compound of Formula 1 or any one of Embodiments 1 through 21 wherein R^{4a} and R^{4b} are each independently C₁-C₂ alkyl or C₁-C₂ haloalkyl.

Embodiment 23. A compound of Embodiment 22 wherein R^{4a} and R^{4b} are each methyl.

Embodiment 24. A compound of Formula 1 or any one of Embodiments 1 through 23 wherein each R⁵ is independently halogen, cyano, C₁-C₂ alkyl, C₁-C₂ alkoxy or C₁-C₂ haloalkyl.

Embodiment 25. A compound of Embodiment 24 wherein each R⁵ is independently Cl, F, methyl or methoxy.

Embodiment 26. A compound of Embodiment 25 wherein each R⁵ is Cl.

Embodiment 27. A compound of Formula 1 or any one of Embodiments 1 through 26 wherein n is 0 or 1.

Embodiment 28. A compound of Embodiment 27 wherein n is 0.

Embodiments of this invention, including Embodiments 1-28 above as well as any other embodiments described herein, can be combined in any manner, and the descriptions of variables in the embodiments pertain not only to the compounds of Formula 1 but also to the starting compounds and intermediate compounds useful for preparing the compounds of Formula 1. In addition, embodiments of this invention, including Embodiments 1-28 above as well as any other embodiments described herein, and any combination thereof, pertain to the compositions and methods of the present invention.

Combinations of Embodiments 1-28 are illustrated by:

Embodiment A1. A compound of Formula 1 wherein
each W is O;
R¹ and R² are each independently H, halogen, C₁-C₄ alkyl, C₂-C₄ alkenyl, C₁-C₄ haloalkyl, C₁-C₄ alkoxy, C₂-C₄ alkylcarbonyl, C₁-C₄ hydroxyalkyl or C₂-C₄ cyanoalkyl;
each R³ is independently halogen, cyano, C₁-C₃ alkyl, C₁-C₃ haloalkyl, C₁-C₃ alkoxy, C₁-C₃ haloalkoxy or C₁-C₃ alkylthio;
R^{4a} and R^{4b} are each methyl;
each R⁵ is independently halogen, cyano, C₁-C₂ alkyl, C₁-C₂ alkoxy or C₁-C₂ haloalkyl;
m is 2 or 3; and
n is 0 or 1.

Embodiment A2. A compound of Embodiment A1 wherein
R¹ and R² are each independently H, halogen, C₁-C₂ alkyl, C₂ alkenyl, C₁-C₂ alkoxy, C₂ alkylcarbonyl or C₁-C₃ hydroxyalkyl;
each R³ is independently Cl, F, cyano, methyl, methoxy or methylthio; and
each R⁵ is independently Cl, F, methyl or methoxy.

Embodiment A3. A compound of Embodiment A2 wherein
R¹ and R² are each independently H, Br, Cl, methyl, C₂ alkenyl or methoxy;
each R³ is independently Cl, F, methyl or methoxy; and
n is 0.

Embodiment A4. A compound of Embodiment A3 wherein
R¹ and R² are each independently Cl or methyl; and
at least one R³ substituent is attached at an ortho position.

Embodiment A5. A compound of Embodiment A4 wherein
two R³ substituents are attached at the ortho positions and one R³ substituent is attached at a meta or the para position; and
m is 3.

Specific embodiments include compounds of Formula 1 selected from the group consisting of:
3-chloro-5-(3,5-dimethoxyphenyl)-6-methyl-4-(2,4,6-trifluorophenyl)pyridazine;
4-(3,5-dimethoxyphenyl)-3,6-dimethyl-5-(2,4,6-trifluorophenyl)pyridazine;
3-chloro-4-(2,6-difluoro-4-methoxyphenyl)-5-(3,5-dimethoxyphenyl)-6-methylpyridazine;
4-(2,6-difluoro-4-methoxyphenyl)-5-(3,5-dimethoxyphenyl)-3,6-dimethylpyridazine;
3-chloro-5-(3,5-dimethoxyphenyl)-4-(2,4,6-trifluorophenyl)pyridazine;
5-(3,5-dimethoxyphenyl)-3-methyl-4-(2,4,6-trifluorophenyl)pyridazine;
3-chloro-5-(3,5-dimethoxyphenyl)-6-methyl-4-(2,3,6-trifluorophenyl)pyridazine;
4-(3,5-dimethoxyphenyl)-3,6-dimethyl-5-(2,3,6-trifluorophenyl)pyridazine; and
3-chloro-4-(3,5-dimethoxyphenyl)-6-methyl-5-(2,4,6-trifluorophenyl)pyridazine.

Of note are compounds of Formula 1 including geometric and stereoisomers, N-oxides, and salts thereof (including but not limited to Embodiments 1-28 and A1-A5 above) wherein R¹ and R² are each independently H, halogen, cyano, C₁-C₆ alkyl, C₂-C₆ alkenyl, C₂-C₆ alkynyl, C₃-C₆ cycloalkyl, C₁-C₆ haloalkyl, C₂-C₆ haloalkenyl, C₁-C₆ hydroxyalkyl, C₁-C₆ alkoxy, C₁-C₆ haloalkoxy, C₁-C₆ alkylthio, C₁-C₆ haloalkylthio, C₂-C₆ alkylcarbonyl or C₂-C₆ alkoxycarbonyl.

This invention provides a fungicidal composition comprising a compound of Formula 1 (including all stereoisomers, N-oxides, and salts thereof), and at least one other fungicide. Of note as embodiments of such compositions are compositions comprising a compound corresponding to any of the compound embodiments described above.

This invention provides a fungicidal composition comprising a compound of Formula 1 (including all stereoisomers, N-oxides, and salts thereof) (i.e. in a fungicidally effective amount), and at least one additional component selected from the group consisting of surfactants, solid diluents and liquid diluents. Of note as embodiments of such compositions are compositions comprising a compound corresponding to any of the compound embodiments described above.

This invention provides a method for controlling plant diseases caused by fungal plant pathogens comprising applying to the plant or portion thereof, or to the plant seed, a fungicidally effective amount of a compound of Formula 1 (including all stereoisomers, N-oxides, and salts thereof). Of note as an embodiment of such methods are methods comprising applying a fungicidally effective amount of a compound corresponding to any of the compound embodiments describe above. Of particular note are embodiments where the compounds are applied as compositions of this invention.

One or more of the following methods and variations as described in Schemes 1-8 can be used to prepare the compounds of Formula 1. The definitions of R¹, R², R³, R^{4a}, R^{4b}, R⁵, W, m and n in the compounds of Formulae 1-14 below are as defined above in the Summary of the Invention unless otherwise noted. Compounds of Formula 1a and 1b are various subsets of Formula 1, and all substituents for Formula 1a and 1b are as defined above for Formula 1 unless otherwise noted.

Compounds of Formula 1 wherein R² is halogen can be prepared from corresponding pyridazinones of Formula 2 by treatment with a halogenating reagent as shown in Scheme 1. Suitable halogenating reagents for this method include phosphorus oxyhalides, phosphorus trihalides, phosphorus pentahalides, thionyl chloride, oxalyl chloride, phenylphosphonic dichloride, phosgene and sulfur tetrafluoride. Phosphorus oxyhalides are particularly useful. Suitable solvents for this reaction include, for example, dichloromethane, chloroform, chlorobutane, benzene, xylenes, chlorobenzene, tetrahydrofuran, p-dioxane, acetonitrile, and the like. In many cases the reaction can be carried out without solvent other than the compound of Formula 2 and the halogenating reagent. Optionally, an organic base such as triethylamine, pyridine, N,N-dimethylaniline, and the like can be added. Addition of a catalyst such as N,N-dimethylformamide is also an option. Typical reaction temperatures range from about room temperature (e.g., 20° C.) to 200° C. For representative procedures see Czarnocki et al., Synthesis 2006, 17, 2855-2864; Brana et al., Journal of Medicinal Chemistry 2005, 48, 6843-6854; Liu et al., Journal of Medicinal Chemistry 2007, 50, 3086-3100 and Chan et al., Journal of Medicinal Chemistry 2005, 48, 4420-4431. The method of Scheme 1 is also illustrated in Example 1, Step F and Example 3, Step E.

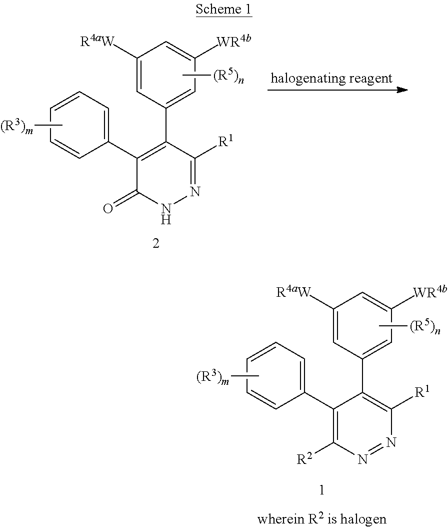

Compounds of Formula 1 wherein R² is halogen (e.g., Br, Cl or I) can be subjected to various nucleophilic and metallation reactions to add substituents or modify existing substituents, and thus provide other functionalized compounds of Formula 1. For example, as shown in Method A of Scheme 2, compounds of Formula 1 wherein R² is halogen (e.g., Cl, Br or I), can be contacted with compounds of formula R²-M¹ in the presence of a suitable palladium, copper or nickel catalyst to produce compounds of Formula 1 wherein R² alkyl, alkenyl, alkynyl, and the like. In this method compounds of formula R²-M¹ are organoboronic acids (e.g., M¹ is B(OH)₂), organoboronic esters (e.g., M¹ is B(—OC(CH₃)₂C(CH₃)₂O—)), organotrifluoroborates (e.g., M¹ is BF₃K), organotin reagents (e.g., M¹ is Sn(n-Bu)₃, Sn(Me)₃), Grignard reagents (e.g., M¹ is MgX¹) or organozinc reagents (e.g., M¹ is ZnX¹) wherein X¹ is Br or Cl. Suitable metal catalysts include, but are not limited to: palladium(II) acetate, palladium(II) chloride, tetrakis(triphenylphosphine)-palladium(0), bis(triphenylphosphine)palladium(II) dichloride, dichloro[1,1′-bis(diphenyl-phosphino)ferrocene]palladium(II), bis(triphenylphosphine)dichloronickel(II) and copper(I) salts (e.g., copper(I) iodide, copper(I) bromide, copper(I) chloride, copper(I) cyanide or copper(I) triflate). Optimal conditions for each reaction will depend on the catalyst used and the counterion attached to the coupling reagent (i.e. M¹), as is understood by one skilled in the art. In some cases the addition of a ligand such as a substituted phosphine or a substituted bisphosphinoalkane promotes reactivity. Also, the presence of a base such as an alkali carbonate, tertiary amine or alkali fluoride is typically necessary for reactions involving organoboron reagents of the formula R²-M¹. For reviews of this type of reaction see: E. Negishi, Handbook of Organopalladium Chemistry for Organic Synthesis, John Wiley and Sons, Inc., New York, 2002; N. Miyaura, Cross-Coupling Reactions: A Practical Guide, Springer, N.Y., 2002; H. C. Brown et al., Organic Synthesis via Boranes, Vol. 3, Aldrich Chemical Co., Milwaukee, Wis., 2002; Suzuki et al., Chemical Review 1995, 95, 2457-2483 and Molander et al., Accounts of Chemical Research 2007, 40, 275-286. Also, Example 2 illustrates the synthesis of a compound of Formula 1 wherein R² is methyl from the corresponding compound wherein R² is chloro.

As shown in Method B of Scheme 2, compounds of Formula 1 wherein R² is alkynyl can be prepared by reaction of the corresponding halide of Formula 1 with a terminal alkyne using Sonogashira reaction conditions. The reaction typically involves the use of two catalysts, a zero-valent palladium complex (or one that can be reduced to Pd(0) in situ) and a halide salt of copper(I). Useful catalysts for this type of transformation include tetrakis-(triphenylphosphine)palladium(0), bis(triphenylphosphine)palladium(II) chloride and dichlorobis(tri-o-tolylphosphine)palladium. Suitable solvents include amines (e.g., triethylamine or diethylamine), or solvents such as tetrahydrofuran, acetonitrile, ethyl acetate and NA-dimethylformamide used in combination with a large excess of a base including, for example, triethylamine, diethylamine, potassium carbonate or cesium carbonate. For leading references see, for example, Campbell, Organocopper Reagents 1994, 217-235; Sonogashira et al., Tetrahedron Letters 1975, 50, 4467-4470 and Chinchilla et al., Chemical Review 2007, 107, 874-922.

As shown in Method C of Scheme 2, compounds of Formula 1 wherein R² is halogen can also undergo nucleophilic displacement reactions to provide compounds of Formula 1 wherein R² is alkoxy, alkylthio, and the like (e.g., displacements with alkoxides and thiolates). Typically these reactions are run in the presence of a suitable base (e.g., sodium hydride, potassium t-butoxide, potassium carbonate or triethylamine), a palladium, nickel or copper catalyst (e.g., tris(dibenzylideneacetone)dipalladium, palladium(II) acetate, bis(1,5-cyclooctadiene)nickel or copper(I) iodide) and optionally a ligand (e.g., 1,1′-bis(diphenylphosphino)ferrocene, 1,3-bis(diphenylphosphino)propane, 2,2′-bis(diphenyl-phosphino)-1,1′-binaphthalene, 1,1′-binaphthalene-2,2′-diol or 1,1,1-tris(hydroxymethyl)-ethane) in a solvent such as methanol, acetonitrile or N,N-dimethylformamide at a temperature ranging from about room temperature to the reflux temperature of the solvent. General procedures for conducting nucleophilic displacements of halogens are known in the art and can be readily adapted to prepare compounds of the present invention. For relevant literature references see, for example, Chen et al., Organic Letters 2006, 8, 5609-5612; Hartwig, Angew. Chem. Int. Ed. 1998, 37(15), 2046-2067 and Buchwald et al., Accounts of Chemical Research 1998, 31(12), 805-818.

As shown in Method D of Scheme 2, reaction of a compound of Formula 1 wherein R² is halogen with a cyanating reagent such as sodium cyanide, potassium cyanide, potassium hexacyanoferrate(II) or sodium hexacyanoferrate(II) provides compounds of Formula 1 wherein R² is nitrile. There are a variety of conditions published in the chemistry literature which can be used for converting a halide of Formula 1 to the corresponding nitrile compound, including copper-catalyzed conditions involving the use of a suitable copper source (e.g., copper(I) iodide), an amine ligand (e.g., N,N′-dimethylethylenediamine) and an iodide salt (e.g., copper(I) iodide, sodium iodide, potassium iodide or zinc iodide). The reaction is typically conducted in a suitable organic solvent such as xylenes, toluene or acetonitrile. For reaction conditions see Buchwald et al., J. Am. Chem. Soc. 2003, 125, 289-2891; Schareina et al., Synlett 2007, 4, 555-558 and Schareina et al., Chem. Eur. 12007, 13, 6249-6254.

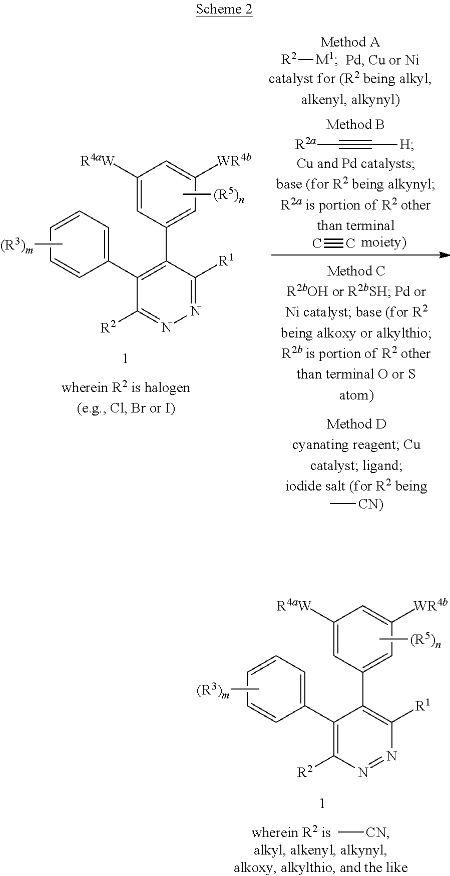

Compounds of Formula 1 wherein R¹ is halogen can be prepared by the two-step synthesis outlined in Scheme 3. In the first step, compounds of Formula 1a (Formula 1 wherein R¹ is H, prepared by the method of Scheme 1) are converted to the corresponding N-oxides of Formula 1b by treatment with an oxidizing reagent such as m-chloroperbenzoic acid (MCPBA) in an appropriate solvent such as chloroform or dichloromethane at a temperature ranging from about 0 to 20° C. Depending on the reaction conditions, isomeric mixtures of 1- and 2-N-oxides can result. Example 4 illustrates the oxidation method of Scheme 3.

Subsequent treatment of a compound of Formula 1b with a halogenating reagent results in displacement of hydrogen with halogen accompanied by loss of the oxide group to provide Formula 1 compounds wherein R¹ is halogen. Halogenating reagents and conditions described for the method of Scheme 1 can be used for the method of Scheme 3. In some cases other functionalities that may be present on compounds of Formula 1b can effect the outcome of the reaction. For example, halogenation can occur on the R² substituent attached to Formula 1b when R² is alkyl, thus forming compounds of Formula 1 wherein R² haloalkyl and R¹ is H. Example 5 illustrates the halogenation method of Scheme 3.

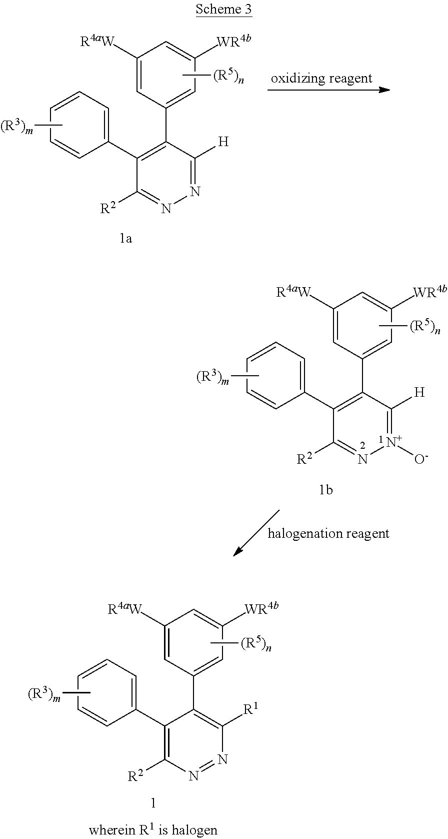

Compounds of Formula 1 wherein R¹ is halogen can be subjected to various nucleophilic and metallation reactions analogous to those described above for Scheme 2 to provide other functionalized compounds of Formula 1. For example, as outlined in Scheme 4, compounds of Formula 1 wherein R¹ is halogen are useful for preparing the corresponding analogs wherein R¹ is methylcarbonyl or hydroxyalkyl. As shown, a compound of Formula 1 wherein R¹ is halogen can be contacted with an organotin reagent such as trimethyl(1-ethoxyethenyl)stannane or tributyl(1-ethoxyethenyl)stannane in the presence of a Pd-catalyzed to provide the 1-methoxy or 1-ethoxyethenyl compounds of Formula 3. Subsequent hydrolysis of Formula 3 provides the methylcarbonyl analogs of Formula 1. The methylcarbonyl analogs of Formula 1 can be treated with an alkyl Grignard reagent in a suitable solvent such as tetrahydrofuran, ether or toluene to obtain compounds of Formula 1 wherein R¹ is hydroxyalkyl. Reactions of this type can be found in the literature; see, for example, Cooke, Journal of Organic Chemistry 1986, 51(6) 951-953. The present Examples 8 and 9 illustrate the method of Scheme 4.

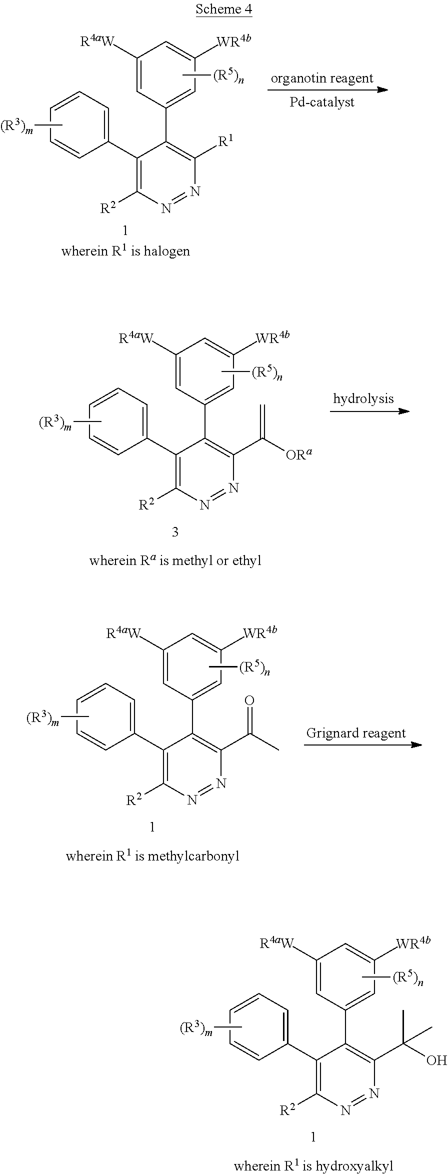

As shown in Scheme 5, intermediates of Formula 2 (shown in Scheme 1), can be synthesized by condensation of furanones of Formula 4 with hydrazine hydrate. The reaction is typically run in a lower alkanol solvent, such as methanol, ethanol or n-butanol at a temperature ranging from about room temperature to the reflux temperature of the solvent. For conditions and variations of this reaction see the following references: PCT Patent Application Publications WO 07/044,796 and WO 98/41511, European Patent Application EP 1916240-A and Piatak et al., Journal of Medicinal Chemistry 1964, 7(5), 590-592. Also, Example 1, Step E and Example 3, Step D illustrate the preparation of a compound of Formula 2.

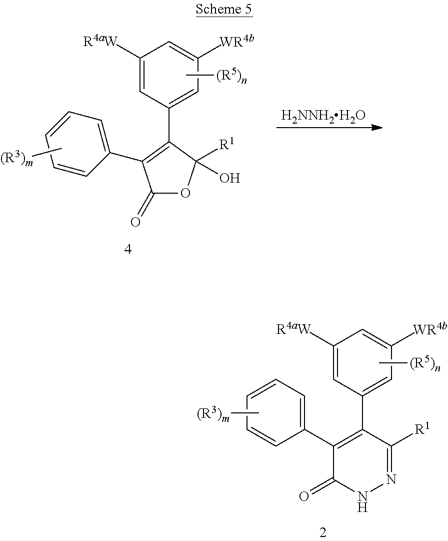

Compounds of Formula 4 can be synthesized by oxidation of furanones of Formula 5 as shown in Scheme 6. The oxidation reaction can be performed by contacting a compound of Formula 5 with an oxygen-containing gas such as air or oxygen, for example by bubbling oxygen or air into a reaction mixture comprising a compound of Formula 5. The reaction is conducted in a suitable solvent such as acetonitrile, ethyl acetate or tetrahydrofuran and optionally in the presence of a catalyst such as activated charcoal or a transition metal such as one comprising palladium, copper or iron. General procedures for conducting oxidations using an oxygen-containing gas are known in the art; see, for example, PCT Patent Application Publications WO 08/049,585 and WO 96/36623; and Nicoll-Griffith et al., Bioorganic and Medicinal Chemistry Letters 2000, 10, 2683-2686. Also, Example 3, Step C illustrates the oxidation method of Scheme 6 using air and activated charcoal. Oxidation of Formula 5 using more potent oxidizers such as 3-chloroperbenzoic acid (MCPBA) in a solvent such chloroform can also be used.

Alternatively, compounds of Formula 5 can be chlorinated or brominated by treatment with N-chlorosuccinimide (NCS) or N-bromosuccinimide (NBS) to give intermediates of Formula 6. The intermediates of Formula 6 can subsequently be hydrolyzed to provide compounds of Formula 4 using a catalytic amount of an acid such as acetic acid in a solvent system such as tetrahydrofuran and water according to the procedure given by Li et al., Bioorganic Medicinal Chemistry Letters 1976, 21, 1839-1842 and the procedure disclosed in PCT Patent Application Publication WO 98/41511. In view of simplicity of operation, low cost of reactants and ease of isolating the desired product, the contact oxidation method using an oxygen-containing gas described in the above paragraph is most advantageous.

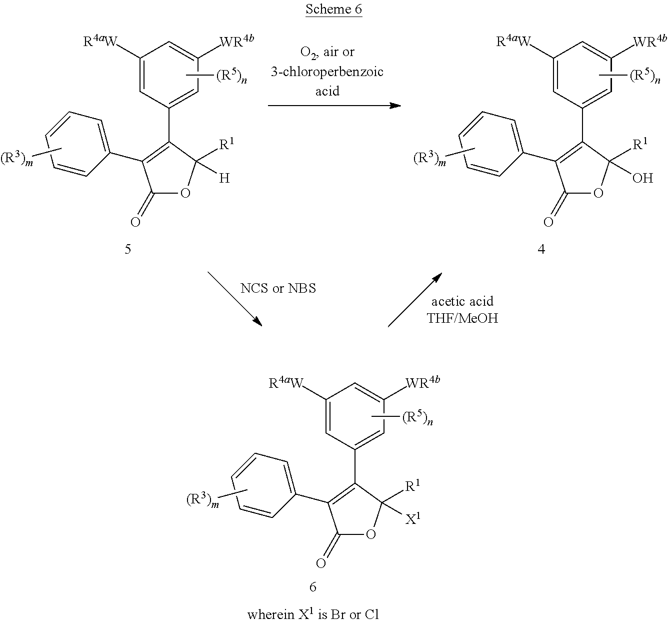

As shown in Scheme 7, the preparation of a compound of Formula 5 can be accomplished by reacting an α-haloketone of Formula 7 with a phenyl acetic acid of Formula 8 in the presence of a suitable base (e.g., a tertiary amine base such as triethylamine or an inorganic base such sodium hydroxide or potassium carbonate) to provide the corresponding ester, which undergoes intramolecular cyclization in the presence of 1,8-diazabicyclo[5.4.0]undec-7-ene (DBU) to provide a compound of Formula 5.

If desired, the cyclization method of Scheme 7 and oxidation method of Scheme 6 can be combined in one reaction vessel such that a compound of Formula 4 is prepared directly from a compound of Formula 7 without isolating Formula 5. Typical reaction conditions involve contacting compounds of Formulae 7, 8 and the base in a solvent such as methanol, dioxane, tetrahydrofuran, acetonitrile, dimethylsulfoxide or NA-dimethylformamide at a temperature between about 5 and 25° C. Preferably the reaction is run using an excess of the base relative to the compounds of Formulae 7 and 8, usually in the range of about 1.5 to about 3 molar equivalents. After formation of the ester (about 8 to 24 h), the reaction mixture is treated with DBU to promote cyclization, and then a stream of air or oxygen is passed through the reaction mixture thus providing compounds of Formula 4. This method is further described in the following references: European Patent Application EP 1916240-A; Black et al., Bioorganic and Medicinal Chemistry Letters 2003, 13, 1195-1198 and Padakanti et al., Tetrahedron Letters 2002, 43, 8715-8719. Present Example 1, Step D illustrates the preparation of a compound of Formula 4 directly from a compound of Formula 7.

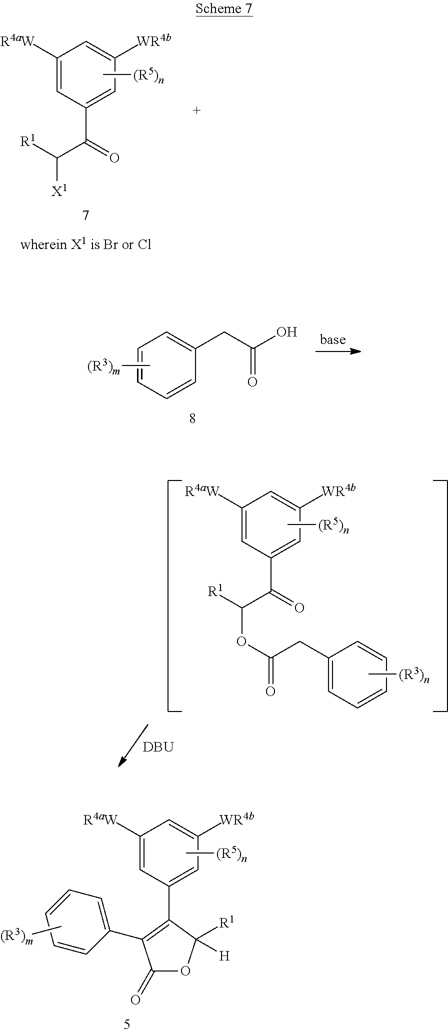

Although the method of Scheme 7 illustrates the reaction of an α-haloketone of Formula 7 and a phenyl acetic acid of Formula 8, one skilled in the art will recognize that depending on the availability of starting materials and/or how other functionalities that may be present in compounds of Formulae 7 and 8 can effect the outcome of the reaction, it may be more advantageous to perform the methods analogous to Schemes 1, 5, 6 and 7 by reacting a phenyl acetic acid analogous to Formula 8 wherein the phenyl ring is substituted with WR^{4a}, WR^{4b} and (R⁵)ₙ and an α-haloketone analogous to Formula 7 containing a phenyl ring substituted by (R³)ₘ to provide compounds analogous to Formulae 5, 4, 2 and 1 wherein R¹ and R² are interchanged, as is illustrated in present Example 6, Step B.

Compounds of Formula 7 are commercial available and can also be prepared from the corresponding ketones by standard halogenation methods known in the art. Particularly useful halogenating reagents for preparing compounds of Formula 7 include elemental halogen (Cl₂, Br₂), N-halosuccinimides (NBS, NCS), copper(II) halides (e.g., CuBr₂, CuCl₂) and pyridinium bromide perbromide. Example 1, Step C, Example 3, Step A and Example 6, Step A illustrate the preparation of α-bromoketones.

In an alternatively method to Scheme 5, intermediates of Formula 2 wherein R¹ is other than halogen can be prepared using the well-known Suzuki coupling reaction as outlined in Scheme 8. In the first step, the N—H nitrogen atom in the compound of Formula 9 is protected prior to the coupling reaction. Nitrogen-protecting groups and methods for protecting nitrogen atoms with these protecting groups are described in Greene, T. W.; Wuts, P. G. M. Protective Groups in Organic Synthesis, 2nd ed.; Wiley: New York, 1991. Metal-catalyzed Suzuki coupling reactions can then be performed to introduce the two phenyl rings onto the pyridazine ring. For preferential displacement of iodo in compounds of Formula 10, the X² group should be less reactive than iodo under coupling conditions, thus allowing for differentiation between the two reactive centers. Use of compounds of Formula 10 wherein X² is Br or Cl often provides optimal selectivity. For typical Suzuki reaction conditions see, for example, Suzuki et al., Chemical Review, 1995, 95, 2457-2483. A wide variety of catalysts are useful for this type of transformation; particularly useful as a catalyst is tetrakis-(triphenylphosphine)palladium(0). Solvents such as tetrahydrofuran, acetonitrile, diethyl ether or dioxane are suitable. The protecting group on Formula 14 can be removed by standard deprotection conditions to give compounds of Formula 2.

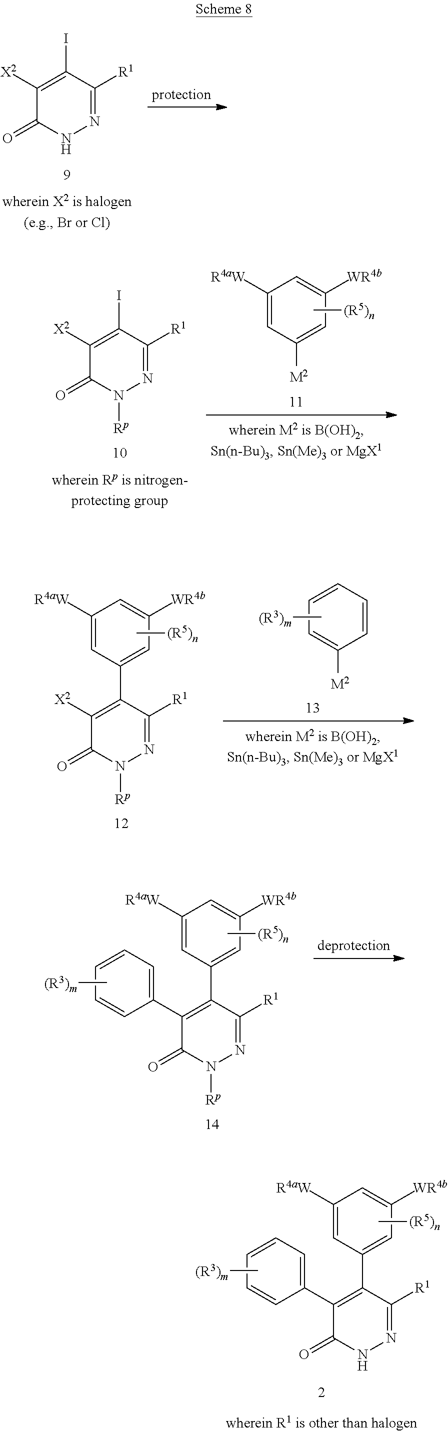

One skilled in the art recognizes that because of the symmetry of the pyridazine ring, the order of introduction of the groups R¹, R² and the phenyl rings substituted with WR^{4a}, WR^{4b} and (R⁵)ₙ onto the pyridazine ring can often be reversed through the use of methods analogous to those described for Schemes 1-8. For example, in a method analogous to Scheme 8, the R¹ substituent in the compound of Formula 9 can be replaced by R² and then the protected intermediate reacted first with a compound of Formula 13 and then a compound of Formula 11 to provide, after deprotection, a compound analogous to Formula 2 except that the phenyl rings are interchanged and the R¹ substituent is replaced by R². Compounds of Formula 2 can then undergo halogenation analogous to the method of Scheme 1 to provide compounds of Formula 1 wherein R¹ is halogen.

Furthermore, one skilled in the art recognizes that for some compounds of Formula 1 the substituents (R³)ₘ and/or (R⁵)ₙ may be more conveniently attached to the phenyl rings after forming the central pyridazine ring. For example, compounds of Formula 1 can be prepared using methods analogous to Schemes 1-8, and then reacted with a halogenating reagent to introduce a R³ and/or R⁵. Present Example 7 illustrates the chlorination of a compound of Formula 1 to add the R⁵ substituent 2-chloro to the phenyl ring substituted with WR^{4a} and WR^{4b}.

It is recognized that some reagents and reaction conditions described above for preparing compounds of Formula 1 may not be compatible with certain functionalities present in the intermediates. In these instances, the incorporation of protection/deprotection sequences or functional group interconversions into the synthesis will aid in obtaining the desired products. The use and choice of the protecting groups will be apparent to one skilled in chemical synthesis (see, for example, Greene, T. W.; Wuts, P. G. M. Protective Groups in Organic Synthesis, 2nd ed.; Wiley: New York, 1991). One skilled in the art will recognize that, in some cases, after the introduction of a given reagent as it is depicted in any individual scheme, it may be necessary to perform additional routine synthetic steps not described in detail to complete the synthesis of compounds of Formula 1. One skilled in the art will also recognize that it may be necessary to perform a combination of the steps illustrated in the above schemes in an order other than that implied by the particular sequence presented to prepare the compounds of Formula 1.

One skilled in the art will also recognize that compounds of Formula 1 and the intermediates described herein can be subjected to various electrophilic, nucleophilic, radical, organometallic, oxidation, and reduction reactions to add substituents or modify existing substituents.

Without further elaboration, it is believed that one skilled in the art using the preceding description can utilize the present invention to its fullest extent. The following Examples are, therefore, to be construed as merely illustrative, and not limiting of the disclosure in any way whatsoever. Steps in the following Examples illustrate a procedure for each step in an overall synthetic transformation, and the starting material for each step may not have necessarily been prepared by a particular preparative run whose procedure is described in other Examples or Steps. Percentages are by weight except for chromatographic solvent mixtures or where otherwise indicated. Parts and percentages for chromatographic solvent mixtures are by volume unless otherwise indicated. ¹H NMR spectra are reported in ppm downfield from tetramethylsilane; “s” means singlet, “d” means doublet, “t” means triplet, “q” means quartet, “m” means multiplet, and “br s” means broad singlet.

### Example 1

### Preparation of 3-chloro-5-(3,5-dimethoxyphenyl)-6-methyl-4-(2,4,6-trifluorophenyl)-pyridazine (Compound 8)

### Step A: Preparation of 3,5-dimethoxy-N,N-dimethylbenzamide

To a mixture of N,N-dimethylamine (2 M in tetrahydrofuran, 31 mL, 62 mmol) in dichloromethane (90 mL) at −10° C. was added triethylamine (17.4 mL, 125 mmol), followed by a dropwise addition of 3,5-dimethoxybenzoyl chloride (10 g, 50 mmol) in dichloromethane (40 mL) while maintaining the temperature of the reaction mixture below 10° C. The reaction mixture was allowed to warm to room temperature, stirred for 15 minutes, and then diluted with hydrochloric acid (1 N) and dichloromethane, the layers were separated, and the aqueous layer was extracted with dichloromethane. The combined organic layers were washed with saturated aqueous sodium bicarbonate solution and saturated aqueous sodium chloride solution, dried over magnesium sulfate, filtered and concentrated under reduced pressure to provide the title compound as an oil (8.41 g).

¹H NMR (CDCl₃): δ 6.5 (s, 2H), 6.48 (s, 1H), 3.79 (s, 6H), 3.09 (br s, 3H), 2.97 (br s, 3H).

### Step B: Preparation of 1-(3,5-dimethoxyphenyl)-1-propanone

To a mixture of 3,5-dimethoxy-N,N-dimethylbenzamide (i.e. the product of Step A) (8.41 g, 40.19 mmol) in tetrahydrofuran (130 mL) at 0° C. was added ethylmagnesium chloride (2 M in tetrahydrofuran, 60 mL, 121 mmol). The reaction mixture was allowed to warm to room temperature, stiffed for 4 h, and then diluted with hydrochloric acid (1 N, 160 mL) and ethyl acetate, the layers were separated, and the aqueous layer was extracted with ethyl acetate. The combined organic layers were washed with saturated aqueous sodium chloride solution, dried over magnesium sulfate, filtered and concentrated. The resulting oil was purified by silica gel column chromatography (30% ethyl acetate in hexanes as eluant) to provide the title compound as an oil (5.69 g).

¹H NMR (CDCl₃): δ 7.1 (s, 2H), 6.6 (s, 1H), 3.84 (s, 6H), 2.9 (q, 2H), 1.2 (t, 3H).

### Step C: Preparation of 2-bromo-1-(3,5-dimethoxyphenyl)-1-propanone

To a of mixture 1-(3,5-dimethoxyphenyl)-1-propanone (i.e. the product of Step B) (5.69 g, 29.14 mmol) in chloroform (33 mL) and acetonitrile (33 mL) was added copper(II) bromide (13.08 g, 58.6 mmol). The reaction mixture was heated at reflux for 6 h, cooled to room temperature and stirred overnight. The reaction mixture was diluted with saturated aqueous sodium bicarbonate solution and ethyl acetate, and then filtered through a bed of Celite® (diatomaceous filter aid) in a sintered glass frit funnel. The layers were separated and the aqueous layer was extracted with ethyl acetate. The combined organic layers were washed with saturated aqueous sodium chloride solution, dried over magnesium sulfate, filtered and concentrated under reduced pressure to provide the title compound as an oil (8 g).

¹H NMR (CDCl₃): δ 7.15 (s, 2H), 6.6 (s, 1H), 5.2 (q, 1H), 3.84 (s, 6H), 1.89 (d, 3H).

### Step D: Preparation of 4-(3,5-dimethoxyphenyl)-5-hydroxy-5-methyl-3-(2,4,6-trifluorophenyl)-2(5H)-furanone

To a of mixture 2-bromo-1-(3,5-dimethoxyphenyl)-1-propanone (i.e. the product of Step C) (4.16 g, 15.2 mmol) and 2,4,6-trifluorobenzeneacetic acid (2.89 g, 15.20 mmol) in acetonitrile (38 mL) was added triethylamine (3.61 mL, 25.9 mmol). The reaction mixture was stirred overnight, and then 1,8-diazabicyclo[5.4.0]undec-7-ene (DBU) (5.05 mL, 33.5 mmol) was added. After 1 h, air was bubbled below the surface of the reaction mixture for 3 h. The reaction mixture was diluted with hydrochloric acid (1 N) and ethyl acetate, the layers were separated and the aqueous layer was extracted with ethyl acetate (2×). The combined organic layers were washed with saturated aqueous sodium chloride solution, dried over magnesium sulfate, filtered and concentrated under reduced pressure. The resulting material was purified by silica gel column chromatography (20 to 30% gradient of ethyl acetate in hexanes as eluant) to provide the title compound as an oil (2.7 g).

¹H NMR (CDCl₃): δ 6.8 (m, 1H), 6.7-6.6 (m, 3H), 6.5 (s, 1H), 3.68 (s, 6H), 1.81 (s, 3H).

### Step E: Preparation of 5-(3,5-dimethoxyphenyl)-4,5-dihydro-6-methyl-4-(2,4,6-trifluorophenyl)-3(2H)-pyridazinone

To a of mixture 4-(3,5-dimethoxyphenyl)-5-hydroxy-5-methyl-3-(2,4,6-trifluoro-phenyl)-2(5H)-furanone (i.e. the product of Step D) (2.7 g, 7.1 mmol) in n-butanol (17 mL) was added hydrazine monohydrate (0.92 g, 18.5 mmol). The reaction mixture was heated at reflux for 6 h. After cooling to room temperature, the reaction mixture was concentrated under reduced pressure, diluted with toluene and again concentrated to provide the title compound as an oil (1.97 g).

¹H NMR (CDCl₃): δ 6.57 (m, 2H), 6.38 (m, 1H), 6.2 (s, 2H), 3.71 (s, 6H), 2.15 (s, 3H).

### Step F: Preparation of 3-chloro-5-(3,5-dimethoxyphenyl)-6-methyl-4-(2,4,6-trifluoro-phenyl)pyridazine

A mixture of 5-(3,5-dimethoxyphenyl)-4,5-dihydro-6-methyl-4-(2,4,6-trifluoro-phenyl)-3(2H)-pyridazinone (i.e. the product of Step E) (1.97 g, 5.24 mmol) and phosphorus oxychloride (30 mL) was heated at reflux for 90 minutes. The reaction mixture was concentrated under reduced pressure, diluted with toluene and again concentrated. The resulting material was partitioned between ethyl acetate and saturated aqueous sodium bicarbonate solution, the layers were separated and the aqueous layer was extracted with ethyl acetate. The combined organic layers were washed with saturated aqueous sodium chloride solution, dried over magnesium sulfate, filtered and concentrated under reduced pressure. The resulting material was purified by silica gel column chromatography (10% ethyl acetate in hexanes as eluant) to provide an oil. The oil was triturated with hexanes and filtered to provide the title compound, a compound of the present invention, as a solid (348 mg).

¹H NMR (CDCl₃): δ 6.63 (t, 2H), 6.38 (s, 1H), 6.19 (s, 2H), 3.71 (s, 6H), 2.57 (s, 3H).

### Example 2

### Preparation of 4-(3,5-dimethoxyphenyl)-3,6-dimethyl-5-(2,4,6-trifluorophenyl)pyridazine (Compound 9)

To a mixture of 3-chloro-5-(3,5-dimethoxyphenyl)-6-methyl-4-(2,4,6-trifluorophenyl)-pyridazine (i.e. the product of Example 1) (100 mg, 0.25 mmol) in p-dioxane (1.3 mL) was added dichloro[1,1′-bis(diphenylphosphino)ferrocene]palladium(II) dichloromethane complex (1:1) (21 mg, 0.025 mmol), cesium carbonate (248 mg, 0.76 mmol), 2,4,6-trimethylboroxine (36 μL, 0.25 mmol) and water (0.12 mL). The reaction mixture was heated at reflux for 3 h, and then allowed to stand overnight at room temperature. The reaction mixture was partitioned between ethyl acetate and water, the layers were separated and the aqueous layer was extracted with ethyl acetate. The combined organic layers were washed with saturated aqueous N′-1,2-ethanediylbis[N-(carboxymethyl)glycine (EDTA) solution and saturated aqueous sodium chloride solution, dried over magnesium sulfate, filtered and concentrated under reduced pressure. The resulting material was purified by silica gel column chromatography (30% ethyl acetate in hexanes as eluant) to provide an oil. The oil was triturated with hexanes and filtered to provide the title compound, a compound of the present invention, as a solid (22 mg).

¹H NMR (CDCl₃): δ 6.6 (t, 2H), 6.3 (s, 1H), 6.18 (s, 2H), 3.71 (s, 6H), 2.54 (s, 3H), 2.49 (s, 3H).

### Example 3

### Preparation of 3-chloro-5-(3,5-dimethoxyphenyl)-4-(2,4,6-trifluorophenyl)pyridazine (Compound 5)

### Step A: Preparation of 2-bromo-1-(3,5-dimethoxyphenyl)ethanone

To a mixture of 1-(3,5-dimethoxyphenyl)ethanone (10 g, 55 mmol) in dichloromethane (140 mL) was added pyridinium bromide perbromide (19.75 g, 55.49 mmol). After stiffing overnight, the reaction mixture was diluted with water, the layers were separated and the aqueous layer was extracted with dichloromethane. The combined organic layers were washed with saturated aqueous bisulfite solution and saturated aqueous sodium chloride solution, dried over magnesium sulfate, filtered and concentrated under reduced pressure. The resulting oil was diluted with diethyl ether/hexanes and filtered to provide the title compound as a yellow solid (5.97 g). The diethyl ether/hexanes filtrate was concentrated to provide more of the title compound as an oil (9.47 g).

¹H NMR (CDCl₃): δ 7.1 (s, 2H), 6.6 (s, 1H), 4.42 (s, 2H), 3.84 (s, 6H).

### Step B: Preparation of 4-(3,5-dimethoxyphenyl)-3-(2,4,6-trifluorophenyl)-2(5H)-furanone

To a mixture of 2-bromo-1-(3,5-dimethoxyphenyl)ethanone (i.e. the product of Step A) (14.37 g, 55.46 mmol) and 2,4,6-trifluorobenzeneacetic acid (10.54 g, 55.46 mmol) in acetonitrile (140 mL) was added triethylamine (13.14 mL, 94.28 mmol). After stiffing overnight, the reaction mixture was cooled to −10° C., and then DBU (18.39 mL, 122.0 mmol) was added while maintaining the temperature of the mixture below 0° C. After stirring for 95 minutes, the reaction mixture was diluted with hydrochloric acid (1 N) and ethyl acetate, the layers were separated and the aqueous layer was extracted with ethyl acetate. The combined organic layers were washed with saturated aqueous sodium chloride solution, dried over magnesium sulfate, filtered and concentrated under reduced pressure. The resulting material was purified by silica gel column chromatography (5 to 40% gradient of ethyl acetate in hexanes as eluant) to provide the title compound as a yellow solid (7.17 g).

¹H NMR (CDCl₃): δ 6.7 (t, 2H), 6.5 (s, 1H), 6.4 (s, 2H), 5.28 (s, 2H), 3.68 (s, 6H).

### Step C: Preparation of 4-(3,5-dimethoxyphenyl)-5-hydroxy-3-(2,4,6-trifluorophenyl)-2(5H)-furanone

A mixture of 4-(3,5-dimethoxyphenyl)-3-(2,4,6-trifluorophenyl)-2(5H)-furanone (i.e. the product of Step B) (7.17 g, 20.5 mmol) and Darco® G-60 (activated charcoal powder, −100 mesh particle size) in ethyl acetate (150 mL) was stirred under air overnight. The reaction mixture was filtered through a bed of Celite® (diatomaceous filter aid) on a sintered glass frit funnel, the Celite® was rinsed with hot ethyl acetate and the filtrate was concentrated under reduced pressure. The resulting material was purified by silica gel column chromatography (5 to 40% gradient of ethyl acetate in hexanes as eluant) to provide the title compound as a yellow solid (3.98 g).

¹H NMR (CDCl₃): δ 6.7 (m, 2H), 6.58 (s, 2H), 6.5 (s, 1H), 3.68 (s, 6H).

### Step D: Preparation of 5-(3,5-dimethoxyphenyl)-4,5-dihydro-4-(2,4,6-trifluoro-phenyl)-3(2H)-pyridazone

To a mixture of 4-(3,5-dimethoxyphenyl)-5-hydroxy-3-(2,4,6-trifluorophenyl)-2 (5H)-furanone (i.e. the product of Step C) (3.4 g, 9.4 mmol) in n-butanol (23 mL) was added hydrazine monohydrate (1.18 mL, 24.4 mmol). The reaction mixture was heated at reflux for 3 h, and then cooled to room temperature and concentrated under reduced pressure. The resulting material was dissolved in dichloromethane and again concentrated to provide the title compound as an oil (3.7 g).

¹H NMR (CDCl₃): δ 7.9 (s, 1H), 6.6 (t, 2H), 6.4 (s, 1H), 6.3 (s, 2H), 3.6 (s, 6H).

### Step E: Preparation of 3-chloro-5-(3,5-dimethoxyphenyl)-4-(2,4,6-trifluorophenyl)pyridazine

A mixture of 5-(3,5-dimethoxyphenyl)-4,5-dihydro-4-(2,4,6-trifluorophenyl)-3 (2H)-pyridazone (i.e. the product of Step D) (3.4 g, 9.4 mmol) and phosphorus oxychloride (40 mL) was heated at reflux for 3.5 h. The reaction mixture was concentrated under reduced pressure, diluted with toluene and again concentrated. The resulting material was partitioned between ethyl acetate and saturated aqueous sodium bicarbonate solution, the layers were separated and the aqueous layer was extracted with ethyl acetate. The combined organic layers were washed with saturated aqueous sodium chloride solution, dried over magnesium sulfate, filtered and concentrated under reduced pressure. The resulting material was purified by silica gel column chromatography (5 to 30% gradient of ethyl acetate in hexanes as eluant) to provide the title compound, a compound of the present invention, as a solid (0.62 g).

¹H NMR (CDCl₃): δ 9.2 (s, 1H), 6.7 (t, 2H), 6.4 (s, 1H), 6.29 (s, 2H), 3.7 (s, 6H).

### Example 4

### Preparation of 5-(3,5-dimethoxyphenyl)-3-methyl-4-(2,4,6-trifluorophenyl)pyridazine 1-oxide and 5-(3,5-dimethoxyphenyl)-3-methyl-4-(2,4,6-trifluorophenyl)-pyridazine 2-oxide (Compound 22, 1- and 2-N-Oxide Mixture)

To a mixture of 5-(3,5-dimethoxyphenyl)-3-methyl-4-(2,4,6-trifluorophenyl)pyridazine (prepared from 3-chloro-5-(3,5-dimethoxyphenyl)-4-(2,4,6-trifluorophenyl)pyridazine analogous to the procedure of Example 2) (0.29 g, 0.81 mmol) in dichloromethane (5 mL) was added 3-chlorobenzenecarboperoxoic acid (MCPBA) (77%, 234 mg, 1.04 mmol). After stirring overnight, the reaction mixture was diluted with saturated aqueous sodium bisulfite solution and dichloromethane, the layers were separated and the aqueous layer was extracted with dichloromethane. The combined organic layers were washed with saturated aqueous sodium bicarbonate solution (2×) and saturated aqueous sodium chloride solution, dried over magnesium sulfate, filtered and concentrated under reduced pressure to provide the title compounds, compounds of the present invention, as a solid (0.44 g).

¹H NMR (CDCl₃): δ 8.4 (s, 1H), 8.1 (s, 1H), 6.7 (m, 4H), 6.42 (s, 1H), 6.40 (s, 1H), 6.22 (d, 2H) 6.20 (d, 2H), 3.69 (s, 6H), 3.69 (s, 6H) 2.37 (s, 3H), 2.37 (s, 3H).

### Example 5

### Preparation of 3-(chloromethyl)-5-(3,5-dimethoxyphenyl)-4-(2,4,6-trifluorophenyl)-pyridazine (Compound 7) and 3-chloro-4-(3,5-dimethoxyphenyl)-6-methyl-5-(2,4,6-trifluorophenyl)pyridazine (Compound 12)

To a mixture of 5-(3,5-dimethoxyphenyl)-3-methyl-4-(2,4,6-trifluorophenyl)pyridazine 1-oxide and 5-(3,5-dimethoxyphenyl)-3-methyl-4-(2,4,6-trifluorophenyl)pyridazine 2-oxide (i.e. the products of Example 4) (302 mg, 0.804 mmol) was added phosphorus oxychloride (6 mL). The reaction mixture was heated at reflux for 2 h, concentrated under reduced pressure, diluted with toluene and again concentrated. The resulting material was partitioned between ethyl acetate and saturated aqueous sodium bicarbonate solution, the layers were separated and the aqueous layer was extracted with ethyl acetate. The combined organic layers were washed with saturated aqueous sodium chloride solution, dried over magnesium sulfate, filtered and concentrated under reduced pressure. The resulting material was purified by silica gel column chromatography (20% ethyl acetate in hexanes as eluant) to provide 3-(chloromethyl)-5-(3,5-dimethoxyphenyl)-4-(2,4,6-trifluorophenyl)pyridazine, a compound of the present invention, as an oil (0.2 g) and 3-chloro-4-(3,5-dimethoxyphenyl)-6-methyl-5-(2,4,6-trifluorophenyl)pyridazine, a compound of the present invention, as a solid (0.2 g).

¹H NMR (CDCl₃) (Compound 7): δ 9.25 (s, 1H), 6.7 (t, 2H), 6.42 (s, 1H), 6.29 (s, 2H), 4.78 (s, 2H), 3.71 (s, 6H).

¹H NMR (CDCl₃) (Compound 12): δ 6.6 (t, 2H), 6.39 (s, 1H), 6.24 (s, 2H), 3.71 (s, 6H), 2.52 (s, 3H).

### Example 6

### Preparation of 3-chloro-4-(3,5-dimethoxyphenyl)-6-methyl-5-(2,6-difluorophenyl)pyridazine (Compound 4)

### Step A: Preparation of 2-bromo-1-(2,6-difluorophenyl)-1-propanone

To a mixture of 1-(2,6-difluorophenyl)-1-propanone (10 g, 59 mmol) in dichloromethane (150 mL) was added pyridinium bromide perbromide (20.88 g, 58.76 mmol). After stirring overnight, the reaction mixture was diluted with water, the layers were separated and the aqueous layer was extracted with dichloromethane. The combined organic layers were washed with saturated aqueous bisulfite solution and saturated aqueous sodium chloride solution, dried over magnesium sulfate, filtered and concentrated under reduced pressure to provide the title compound as an oil (14.68 g).

¹H NMR (CDCl₃): δ 6.9 (t, 2H), 6.4 (m, 1H), 5.0 (q, 1H), 1.9 (d, 3H).

### Step B: Preparation of 4-(2,6-difluorophenyl)-3-(3,5-dimethoxyphenyl)-5-hydroxy-5-methyl-2(5H)-furanone

To a of mixture 2-bromo-1-(2,6-difluorophenyl)-1-propanone (i.e. the product of Step A) (14.6 g, 58.74 mmol) and 3,5-dimethoxybenzeneacetic acid (11.52 g, 58.7 mmol) in acetonitrile (420 mL) was added triethylamine (13.92 mL, 99.9 mmol). After stirring overnight, DBU (19.48 mL, 129.2 mmol) was added to the reaction mixture. After 1 h, air was bubbled below the surface of the reaction mixture for 3 h. The reaction mixture was diluted with hydrochloric acid (1 N) and ethyl acetate, the layers were separated and the aqueous layer was extracted with ethyl acetate. The combined organic layers were washed with saturated aqueous sodium chloride solution, dried over magnesium sulfate, filtered and concentrated under reduced pressure. The resulting material was purified by silica gel column chromatography (5 to 40% gradient of ethyl acetate in hexanes as eluant) to provide the title compound as an oil (7.98 g).

¹H NMR (CDCl₃): δ 7.39 (m, 1H), 6.9 (t, 2H), 6.6 (s, 2H), 6.4 (s, 2H), 3.78 (s, 6H), 2.0 (s, 2H).

### Step C: Preparation of 5-(2,6-difluorophenyl)-4-(3,5-dimethoxyphenyl)-4,5-dihydro-6-methyl-3 (2H)-pyridazinone

To a of mixture 4-(3,5-dimethoxyphenyl)-5-hydroxy-5-methyl-3-(2,4,6-trifluorophenyl)-2(5H)-furanone (i.e. the product of Step B) (7.98 g, 22.0 mmol) in n-butanol (55 mL) was added hydrazine monohydrate (2.78 mL, 57.3 mmol). The reaction mixture was heated at reflux for 3 h. After cooling to room temperature, the reaction mixture was concentrated under reduced pressure, diluted with toluene and again concentrated to provide the title compound as an oil (6.6 g).

### Step D: Preparation of 3-chloro-4-(3,5-dimethoxyphenyl)-6-methyl-5-(2,6-difluorophenyl)pyridazine

A mixture of 5-(2,6-difluorophenyl)-4-(3,5-dimethoxyphenyl)-4,5-dihydro-6-methyl-3(2H)-pyridazinone (i.e. the product of Step C) (6.62 g, 18.47 mmol) and phosphorus oxychloride (55 mL) was heated at reflux for 2 h. The reaction mixture was concentrated under reduced pressure, diluted with toluene and again concentrated. The resulting material was partitioned between ethyl acetate and saturated aqueous sodium bicarbonate solution, the layers were separated and the aqueous layer was extracted with dichloromethane. The combined organic layers were washed with saturated aqueous sodium chloride solution, dried over magnesium sulfate, filtered and concentrated under reduced pressure. The resulting material was purified by silica gel column chromatography (5 to 40% gradient of ethyl acetate in hexanes as eluant) to provide the title compound, a compound of the present invention, as an oil (0.41 g).

¹H NMR (CDCl₃): δ 7.3 (m, 1H), 6.8 (m, 2H), 6.3 (s, 1H), 6.2 (s, 2H), 3.69 (s, 6H), 2.5 (s, 3H).

### Example 7

### Preparation of 3-chloro-5-(2-chloro-3,5-dimethoxyphenyl)-6-methyl-4-(2,4,6-trifluoro-phenyl)pyridazine (Compound 23)

To a mixture of 3-chloro-5-(3,5-dimethoxyphenyl)-6-methyl-4-(2,4,6-trifluorophenyl)-pyridazine (i.e. the product of Example 1) (100 mg, 0.25 mmol) in carbon tetrachloride (3 mL) was added N-chlorosuccinimide (40 mg, 0.30 mmol) and 2,2′-(1,2-diazenediyl)bis[2-methyl-propanenitrile (AIBN) (catalytic amount). The reaction mixture was heated at 60° C. overnight. After cooling to room temperature, the reaction mixture was diluted with water and ethyl acetate and the layers were separated. The organic layer was washed with saturated aqueous sodium chloride solution, dried over magnesium sulfate, filtered and concentrated under reduced pressure. The resulting material was purified by flash chromatography on a silica gel (5 g), Varian Bond Elute SI® column (10% ethyl acetate in hexanes as eluant) to give a white solid. The resulting white solid was diluted with diethyl ether/hexanes and filtered to provide the title compound, a compound of the present invention, as a white solid (59 g).

¹H NMR (CDCl₃): δ 6.6 (m, 2H), 6.46 (d, 1H), 6.20 (s, 1H), 3.85 (s, 3H), 3.72 (s, 3H), 2.52 (s, 3H).

### Example 8

### Preparation of 1-[4-(3,5-dimethoxyphenyl)-6-methyl-5-(2,4,6-trifluorophenyl)-3-pyridazinyl]ethanone (Compound 20)

### Step A: Preparation of 4-(3,5-dimethoxypehnyl)-3-(1-ethoxyethenyl)-6-methyl-5-(2,4,6-trifluorophenyl)pyridazine

To a mixture of 3-chloro-4-(3,5-dimethoxyphenyl)-6-methyl-5-(2,4,6-trifluorophenyl)-pyridazine (i.e. the product of Example 5, Compound 12) (0.39 g, 1.06 mmol) in N,N-dimethylformamide (12 mL) was added tributyl(1-ethoxyethenyl)stannane (0.5 g, 1.4 mmol) and dichlorobis(triphenylphosphine)palladium (50 mg, 0.07 mmol). The reaction mixture was heated at 80° C. overnight, and then cooled to room temperature and a solution of potassium fluoride (4 g) in water and ethyl acetate was added. After stirring for 1 h, the layers were separated and the aqueous layer was extracted with ethyl acetate. The combined organic layers were washed with water (3×), saturated aqueous sodium chloride solution, dried over magnesium sulfate, filtered and concentrated under reduced pressure to provide the title compound as an oil (0.5 g).

¹H NMR (CDCl₃): δ 6.6 (t, 2H), 6.3 (s, 1H), 6.2 (d, 2H), 4.8 (d, 1H), 4.4 (d, 1H), 3.67 (s, 6H), 3.5 (q, 2H), 2.54 (s, 3H), 1.2 (t, 3H).

### Step B: Preparation of 1-[4-(3,5-dimethoxyphenyl)-6-methyl-5-(2,4,6-trifluorophenyl)-3-pyridazinyl]ethanone

To a mixture of 4-(3,5-dimethoxypehnyl)-3-(1-ethoxyethenyl)-6-methyl-5-(2,4,6-trifluorophenyl)pyridazine (i.e. the product of Step A) (0.45 g, 1.04 mmol) in acetone (5 mL) was added hydrochloric acid (1 N, 1.5 mL). After stirring overnight, the reaction mixture was diluted with saturated sodium bicarbonate solution, the layers were separated and the aqueous layer was extracted with ethyl acetate (2×). The combined organic layers were washed with saturated aqueous sodium chloride solution, dried over magnesium sulfate, filtered and concentrated under reduced pressure. The resulting oil was purified by flash chromatography on a silica gel (5 g), Varian Bond Elute SIC) column (30% ethyl acetate in hexanes as eluant). The resulting white solid was diluted with diethyl ether/hexanes and filtered to provide the title compound, a compound of the present invention, as a yellow solid (0.34 g).

¹H NMR (CDCl₃): δ 6.6 (t, 2H), 6.3 (m, 1H), 6.14 (d, 6H), 6.2 (s, 2H), 3.68 (s, 6H), 2.74 (s, 3H), 2.60 (s, 3H).

### Example 9

### Preparation of 4-(3,5-dimethoxyphenyl)-α,α,6-trimethyl-5-(2,4,6-trifluorophenyl)-3-pyridazinemethanol (Compound 21)

To a mixture of 1-[4-(3,5-dimethoxyphenyl)-6-methyl-5-(2,4,6-trifluorophenyl)-3-pyridazinyl]ethanone (i.e. the product of Example 8) (0.20 g, 0.49 mmol) in tetrahydrofuran (6 mL) at −78° C. was added methyl magnesium chloride (3 M in tetrahydrofuran, 0.5 mL, 1.5 mmol). After the addition was complete, stirring was continued for 1 h at −70° C., and then the reaction mixture was allowed to warm to room temperature. The reaction mixture was then diluted with hydrochloric acid (1 N, 15 mL) and ethyl acetate, the layers were separated and the aqueous layer was extracted with ethyl acetate. The combined organic layers were washed with saturated aqueous sodium chloride solution, dried over magnesium sulfate, filtered and concentrated under reduced pressure to give an oil (0.25 g). The resulting oil was purified by flash chromatography on a silica gel (5 g), Varian Bond Elute SI® column (10 to 30% gradient of ethyl acetate in hexanes as eluant) to give an oil. The resulting oil was diluted with diethyl ether/hexanes and filtered to provide the title compound, a compound of the present invention, as a yellow solid (55 mg).

¹H NMR (CDCl₃): δ 6.6 (t, 2H), 6.3 (m, 1H), 6.2 (d, 2H), 5.79 (br s, 1H), 3.69 (s, 6H), 2.51 (s, 3H), 1.42 (s, 6H).

### Example 10

### Preparation of 3-chloro-6-(chloromethyl)-4-(2,6-difluorophenyl)-5-(3,5-dimethoxyphenyl)-pyridazine (Compound 26)

To phosphorus oxychloride (14 mL) was added 3-chloro-4-(2,6-difluorophenyl)-5-(3,5-dimethoxyphenyl)-6-methyl-1-oxide (prepared from 3-chloro-4-(2,6-difluorophenyl)-5-(3,5-dimethoxyphenyl)-6-methylpyridazine analogous to the procedure of Example 4) (0.72 g, 1.85 mmol). The reaction mixture was heated at reflux for 2 h, concentrated under reduced pressure, diluted with toluene and again concentrated. The resulting material was partitioned between ethyl acetate and saturated aqueous sodium bicarbonate solution, the layers were separated and the aqueous layer was extracted with ethyl acetate. The combined organic layers were washed with saturated aqueous sodium chloride solution, dried over magnesium sulfate, filtered and concentrated under reduced pressure. The resulting material was purified by flash chromatography on a silica gel (10 g), Varian Bond Elute SI® column (20% ethyl acetate in hexanes as eluant). The resulting solid was diluted with hexanes and filtered to provide the title compound, a compound of the present invention, as a solid (0.27 g).

¹H NMR (CDCl₃): δ 7.3 (m, 1H), 6.8 (t, 2H), 6.4 (s, 1H), 6.3 (s, 2H), 4.7 (s, 2H), 3.7 (s, 6H).

### Example 11

### Preparation of 6-chloro-5-(2,6-difluorophenyl)-4-(3,5-dimethoxyphenyl)-3-pyridazine-acetonitrile (Compound 27)

To a mixture of 3-chloro-6-(chloromethyl)-4-(2,6-difluorophenyl)-5-(3,5-dimethoxy-phenyl)pyridazine (i.e. the product of Example 10) (100 g, 0.24 mmol) in methanol (2 mL) was added sodium cyanide (12 mg, 0.24 mmol). The reaction mixture was heated at 60° C. for 4 h. After cooling to room temperature, the reaction mixture was then diluted with water and dichloromethane, the layers were separated and the aqueous layer was extracted with dichloromethane. The combined organic layers were washed with saturated aqueous sodium chloride solution, dried over magnesium sulfate, filtered and concentrated under reduced pressure to give an oil (0.15 g). The resulting oil was purified by flash chromatography on a silica gel (5 g), Varian Bond Elute SI® column (20% ethyl acetate in hexanes as eluant) to provide the title compound, a compound of the present invention, as a solid (60 mg).

¹H NMR (CDCl₃): δ 7.3 (m, 1H), 6.8 (t, 2H), 6.4 (s, 1H), 6.26 (s, 2H), 3.95 (s, 2H), 3.71 (s, 6H).

### Example 12

### Preparation of 4-(2,6-difluorophenyl)-3-fluoro-5-(3,5-dimethoxyphenyl)-6-methylpyridazine (Compound 33)

To a mixture of 3-chloro-4-(2,6-difluorophenyl)-5-(3,5-dimethoxyphenyl)-6-methyl-pyridazine (prepared from 5-(3,5-dimethoxyphenyl)-4,5-dihydro-6-methyl-4-(2,6-trifluoro-phenyl)-3(2H)-pyridazinone analogous to the procedure of Example 1) (1.2 g, 3.19 mmol) in dimethyl sulfoxide (10 mL) was added 18-crown-6 (0.92 mg, 3.51 mmol) and potassium fluoride (0.55 mg, 9.57 mmol). The reaction mixture was heated in a sealed vessel for 36 h at 140° C. After cooling to room temperature, the reaction mixture was then diluted with water, the layers were separated and the aqueous layer was extracted with ethyl acetate (3×). The combined organic layers were washed with saturated aqueous sodium chloride solution, dried over sodium sulfate, filtered and concentrated under reduced pressure. The resulting material was purified by silica gel chromatography to provide the title compound, a compound of the present invention, as a white solid (500 mg).

¹H NMR (CDCl₃): δ 7.3 (m, 1H), 6.8 (t, 2H), 6.4 (s, 1H), 6.26 (s, 2H), 3.95 (s, 2H), 3.71 (s, 6H).

By the procedures described herein together with methods known in the art, the following compounds of Tables 1-3 can be prepared. The following abbreviations are used in the Tables which follow: s means secondary, n means normal, i means iso, c means cyclo, Me means methyl, Et means ethyl, Pr means propyl, i-Pr means isopropyl, Bu means butyl, MeO means methoxy, EtO means ethoxy, MeS means methylthio, CN means cyano and NO₂ means nitro.

TABLE 1

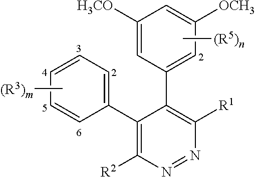

R¹
H
Br
Cl
I
CN
Me
Et
i-Pr
s-Bu
CH₃CH₂C(═CH₂)
(CH₃)₂CHC(═CH₂)
CH₂═CH
CH₃C(═CHCH₃)
CH≡C
c-Pr
c-pentyl
c-hexyl
CH₂F
CH₂Cl
MeO
EtO
CF₃O
MeS
CH₃C(═O)
CH₃CH₂C(═O)
CH₃OC(═O)
CH₃CH₂OC(═O)
CH₃(CH)₂OC(═O)
(CH₃)₂CHOC(═O)
CH₃CH(OH)
CH₃CH₂CH(OH)
(CH₃)₂CHCH(OH)
(CH₃)₂C(OH)
(CN)CH₂
R² is Me;
(R³)ₘ is 2,4,6-tri-F; and n is 0.

The present disclosure also includes Tables 1A through 73A, each of which is constructed the same as Table 1 above except that the row heading in Table 1 (i.e. “R² is Me; (R³)ₘ is 2,4,6-tri-F; and n is 0”) is replaced with the respective row headings shown below. For example, in Table 1A the row heading is “R² is Me; (R³)ₘ is 2,3,4-tri-F; and n is 0”, and R¹ is as defined in Table 1 above. Thus, the first entry in Table 1A specifically discloses 5-(3,5-dimethoxyphenyl)-3-methyl-4-(2,3,4-trifluorophenyl)pyridazine. Tables 2A through 73A are constructed similarly.

| Table | Row Heading |
|---|---|
| 1A | R² is Me; (R³)ₘ is 2,3,4-tri-F; and n is 0. |
| 2A | R² is Me; (R³)ₘ is 2,3,6-tri-F; and n is 0. |
| 3A | R² is Me; (R³)ₘ is 2,4,5-tri-F; and n is 0. |
| 4A | R² is Me; (R³)ₘ is 2,6-di-F, 3-Cl; and n is 0. |
| 5A | R² is Me; (R³)ₘ is 2,6-di-F, 4-Cl; and n is 0. |
| 6A | R² is Me; (R³)ₘ is 2,6-di-F, 3-CN; and n is 0. |
| 7A | R² is Me; (R³)ₘ is 2,6-di-F, 4-CN; and n is 0. |
| 8A | R² is Me; (R³)ₘ is 2,6-di-F, 4-NO₂; and n is 0. |
| 9A | R² is Me; (R³)ₘ is 2,6-di-F, 3-Me; and n is 0. |
| 10A | R² is Me; (R³)ₘ is 2,6-di-F, 4-Me; and n is 0. |
| 11A | R² is Me; (R³)ₘ is 2,6-di-F, 3-MeO; and n is 0. |
| 12A | R² is Me; (R³)ₘ is 2,6-di-F, 4-MeO; and n is 0. |
| 13A | R² is Me; (R³)ₘ is 2,6-di-F, 3-EtO; and n is 0. |
| 14A | R² is Me; (R³)ₘ is 2,6-di-F, 4-EtO; and n is 0. |
| 15A | R² is Me; (R³)ₘ is 2,6-di-F, 4-MeS; and n is 0. |
| 16A | R² is Me; (R³)ₘ is 2,6-di-F, 3-CHF₂O; and n is 0. |
| 17A | R² is Me; (R³)ₘ is 2,6-di-F, 4-CHF₂O; and n is 0. |
| 18A | R² is Me; (R³)ₘ is 2,6-di-F, 4-MeNH; and n is 0. |
| 19A | R² is Me; (R³)ₘ is 2,6-di-F, 3-MeNH; and n is 0. |
| 20A | R² is Me; (R³)ₘ is 2,6-di-F, 4-Me₂N; and n is 0. |
| 21A | R² is Me; (R³)ₘ is 2,6-di-F, 3-Et₂N; and n is 0. |
| 22A | R² is Me; (R³)ₘ is 2,4-di-F, 5-CN; and n is 0. |
| 23A | R² is Me; (R³)ₘ is 2,3-di-Cl, 4-F; and n is 0. |
| 24A | R² is Me; (R³)ₘ is 2,6-di-Cl, 4-F; and n is 0. |
| 25A | R² is Me; (R³)ₘ is 2-Cl, 6-F, 3-MeO; and n is 0. |
| 26A | R² is Me; (R³)ₘ is 2-Cl, 6-F, 4-MeO; and n is 0. |
| 27A | R² is Me; (R³)ₘ is 2-Cl, 6-F, 5-MeO; and n is 0. |
| 28A | R² is Me; (R³)ₘ is 2-Cl, 3,6-di-F; and n is 0. |
| 29A | R² is Me; (R³)ₘ is 2-Cl, 4,6-di-F; and n is 0. |
| 30A | R² is Me; (R³)ₘ is 2,4-di-F; and n is 0. |
| 31A | R² is Me; (R³)ₘ is 2,6-di-F; and n is 0. |
| 32A | R² is Me; (R³)ₘ is 2,4-di-Cl; and n is 0. |
| 33A | R² is Me; (R³)ₘ is 2,5-di-Cl; and n is 0. |
| 34A | R² is Me; (R³)ₘ is 2,6-di-Cl; and n is 0. |
| 35A | R² is Me; (R³)ₘ is 2-F, 6-Me; and n is 0. |
| 36A | R² is Me; (R³)ₘ is 2-F, 6-CF₃; and n is 0. |
| 37A | R² is Me; (R³)ₘ is 2-F, 6-CHF₂O; and n is 0. |
| 38A | R² is Me; (R³)ₘ is 2-Br, 4-F; and n is 0. |
| 39A | R² is Me; (R³)ₘ is 2-Br, 6-F; and n is 0. |
| 40A | R² is Me; (R³)ₘ is 2-Br, 4-MeO; and n is 0. |
| 41A | R² is Me; (R³)ₘ is 2-Cl, 4-F; and n is 0. |
| 42A | R² is Me; (R³)ₘ is 2-Cl, 6-F; and n is 0. |
| 43A | R² is Me; (R³)ₘ is 2-Cl, 6-CN; and n is 0. |
| 44A | R² is Me; (R³)ₘ is 2-Cl, 6-NO₂; and n is 0. |
| 45A | R² is Me; (R³)ₘ is 2-Cl, 4-Me; and n is 0. |
| 46A | R² is Me; (R³)ₘ is 2-Cl, 4-MeO; and n is 0. |
| 47A | R² is Me; (R³)ₘ is 2-Cl, 5-CF₃; and n is 0. |
| 48A | R² is Me; (R³)ₘ is 2-I, 6-F; and n is 0. |
| 49A | R² is Me; (R³)ₘ is 2-NO₂, 4-F; and n is 0. |
| 50A | R² is Me; (R³)ₘ is 2-CN, 6-F; and n is 0. |
| 51A | R² is Me; (R³)ₘ is 2-CF₃, 6-F; and n is 0. |
| 52A | R² is Me; (R³)ₘ is 2-CF₃, 4-MeO; and n is 0. |
| 53A | R² is Me; (R³)ₘ is 2-CHF₂O, 6-F; and n is 0. |
| 54A | R² is Me; (R³)ₘ is 4-MeO; and n is 0. |
| 55A | R² is Me; (R³)ₘ is 4-EtO; and n is 0. |
| 56A | R² is Me; (R³)ₘ is 4-MeS; and n is 0. |
| 57A | R² is Me; (R³)ₘ is 2-CF₃; and n is 0. |
| 58A | R² is Me; (R³)ₘ is 3-MeC(═O); and n is 0. |
| 59A | R² is Me; (R³)ₘ is 4-MeOC(═O); and n is 0. |
| 60A | R² is Me; (R³)ₘ is 4-MeNHC(═O); and n is 0. |
| 61A | R² is Me; (R³)ₘ is 3-Me₂NC(═O); and n is 0. |
| 62A | R² is Me; (R³)ₘ is 2,3,6-tri-F; and (R⁵)ₙ is 2-Cl. |
| 63A | R² is Me; (R³)ₘ is 2,4,6-tri-F; and (R⁵)ₙ is 2-Cl. |
| 64A | R² is Me; (R³)ₘ is 2,6-di-F, 3-Cl; and (R⁵)ₙ is 2-Cl. |
| 65A | R² is Me; (R³)ₘ is 2,6-di-F, 4-Me; and (R⁵)ₙ is 2-Cl. |
| 66A | R² is Me; (R³)ₘ is 2,6-di-F, 4-MeO; and (R⁵)ₙ is 2-Cl. |
| 67A | R² is Me; (R³)ₘ is 2,6-di-Cl, 4-F; and (R⁵)ₙ is 2-Cl. |
| 68A | R² is Me; (R³)ₘ is 2,6-di-F; and (R⁵)ₙ is 2-Cl. |
| 69A | R² is Me; (R³)ₘ is 4-MeO; and (R⁵)ₙ is 2-Cl. |
| 70A | R² is Br; (R³)ₘ is 2,4,6-tri-F; and n is 0. |
| 71A | R² is i-Pr; (R³)ₘ is 2,4,6-tri-F; and n is 0. |
| 72A | R² is c-Pr; (R³)ₘ is 2,4,6-tri-F; and n is 0. |
| 73A | R² is CH₂Cl; (R³)ₘ is 2,4,6-tri-F; and n is 0. |

TABLE 2

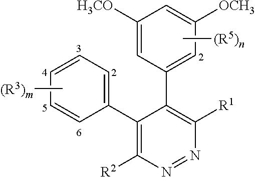

R¹
H
Br
I
CN
Me
Et
i-Pr
s-Bu
CH₃CH₂C(═CH₂)
(CH₃)₂CHC(═CH₂)
CH₂═CH
CH₃C(═CHCH₃)
CH≡C
c-Pr
c-pentyl
c-hexyl
CH₂F
CH₂Cl
MeO
EtO
CF₃O
MeS
CH₃C(═O)
CH₃CH₂C(═O)
CH₃OC(═O)
CH₃CH₂OC(═O)
CH₃(CH)₂OC(═O)
(CH₃)₂CHOC(═O)
CH₃CH(OH)
CH₃CH₂CH(OH)
(CH₃)₂CHCH(OH)
(CH₃)₂C(OH)
(CN)CH₂
R² is Cl;
(R³)ₘ is 2,4,6-tri-F; and n is 0.

The present disclosure also includes Tables 2B through 70B, each of which is constructed the same as Table 2 above except that the row heading in Table 2 (i.e. “R² is Cl; (R³)ₘ is 2,4,6-tri-F; and n is 0”) is replaced with the respective row headings shown below. For example, in Table 2B the row heading is “R² is Cl; (R³)ₘ is 2,3,4-tri-F; and n is 0”, and R¹ is as defined in Table 2 above. Thus, the first entry in Table 2B specifically discloses 5-(3,5-dimethoxyphenyl)-3-chloro-4-(2,3,4-trifluorophenyl)pyridazine. Tables 3B through 70B are constructed similarly.

| Table | Row Heading |
|---|---|
| 2B | R² is Cl; (R³)ₘ is 2,3,4-tri-F; and n is 0. |
| 3B | R² is Cl; (R³)ₘ is 2,3,6-tri-F; and n is 0. |
| 4B | R² is Cl; (R³)ₘ is 2,4,5-tri-F; and n is 0. |
| 5B | R² is Cl; (R³)ₘ is 2,6-di-F, 3-Cl; and n is 0. |
| 6B | R² is Cl; (R³)ₘ is 2,6-di-F, 4-Cl; and n is 0. |
| 7B | R² is Cl; (R³)ₘ is 2,6-di-F, 3-CN; and n is 0. |
| 8B | R² is Cl; (R³)ₘ is 2,6-di-F, 4-CN; and n is 0. |
| 9B | R² is Cl; (R³)ₘ is 2,6-di-F, 4-NO₂; and n is 0. |
| 10B | R² is Cl; (R³)ₘ is 2,6-di-F, 3-Me; and n is 0. |
| 11B | R² is Cl; (R³)ₘ is 2,6-di-F, 4-Me; and n is 0. |
| 12B | R² is Cl; (R³)ₘ is 2,6-di-F, 3-MeO; and n is 0. |
| 13B | R² is Cl; (R³)ₘ is 2,6-di-F, 4-MeO; and n is 0. |
| 14B | R² is Cl; (R³)ₘ is 2,6-di-F, 3-EtO; and n is 0. |
| 15B | R² is Cl; (R³)ₘ is 2,6-di-F, 4-EtO; and n is 0. |
| 16B | R² is Cl; (R³)ₘ is 2,6-di-F, 4-MeS; and n is 0. |
| 17B | R² is Cl; (R³)ₘ is 2,6-di-F, 3-CHF₂O; and n is 0. |
| 18B | R² is Cl; (R³)ₘ is 2,6-di-F, 4-CHF₂O; and n is 0. |
| 19B | R² is Cl; (R³)ₘ is 2,6-di-F, 4-MeNH; and n is 0. |
| 20B | R² is Cl; (R³)ₘ is 2,6-di-F, 3-MeNH; and n is 0. |
| 21B | R² is Cl; (R³)ₘ is 2,6-di-F, 4-Me₂N; and n is 0. |
| 22B | R² is Cl; (R³)ₘ is 2,6-di-F, 3-Et₂N; and n is 0. |
| 23B | R² is Cl; (R³)ₘ is 2,4-di-F, 5-CN; and n is 0. |
| 24B | R² is Cl; (R³)ₘ is 2,3-di-Cl, 4-F; and n is 0. |
| 25B | R² is Cl; (R³)ₘ is 2,6-di-Cl, 4-F; and n is 0. |
| 26B | R² is Cl; (R³)ₘ is 2-Cl, 6-F, 3-MeO; and n is 0. |
| 27B | R² is Cl; (R³)ₘ is 2-Cl, 6-F, 4-MeO; and n is 0. |
| 28B | R² is Cl; (R³)ₘ is 2-Cl, 6-F, 5-MeO; and n is 0. |
| 29B | R² is Cl; (R³)ₘ is 2-Cl, 3,6-di-F; and n is 0. |
| 30B | R² is Cl; (R³)ₘ is 2-Cl, 4,6-di-F; and n is 0. |
| 31B | R² is Cl; (R³)ₘ is 2,4-di-F; and n is 0. |
| 32B | R² is Cl; (R³)ₘ is 2,6-di-F; and n is 0. |
| 33B | R² is Cl; (R³)ₘ is 2,4-di-Cl; and n is 0. |
| 34B | R² is Cl; (R³)ₘ is 2,5-di-Cl; and n is 0. |
| 35B | R² is Cl; (R³)ₘ is 2,6-di-Cl; and n is 0. |
| 36B | R² is Cl; (R³)ₘ is 2-F, 6-Me; and n is 0. |
| 37B | R² is Cl; (R³)ₘ is 2-F, 6-CF₃; and n is 0. |
| 38B | R² is Cl; (R³)ₘ is 2-F, 6-CHF₂O; and n is 0. |
| 39B | R² is Cl; (R³)ₘ is 2-Br, 4-F; and n is 0. |
| 40B | R² is Cl; (R³)ₘ is 2-Br, 6-F; and n is 0. |
| 41B | R² is Cl; (R³)ₘ is 2-Br, 4-MeO; and n is 0. |
| 42B | R² is Cl; (R³)ₘ is 2-Cl, 4-F; and n is 0. |
| 43B | R² is Cl; (R³)ₘ is 2-Cl, 6-F; and n is 0. |
| 44B | R² is Cl; (R³)ₘ is 2-Cl, 6-CN; and n is 0. |
| 45B | R² is Cl; (R³)ₘ is 2-Cl, 6-NO₂; and n is 0. |
| 46B | R² is Cl; (R³)ₘ is 2-Cl, 4-Me; and n is 0. |
| 47B | R² is Cl; (R³)ₘ is 2-Cl, 4-MeO; and n is 0. |
| 48B | R² is Cl; (R³)ₘ is 2-Cl, 5-CF₃; and n is 0. |
| 49B | R² is Cl; (R³)ₘ is 2-I, 6-F; and n is 0. |
| 50B | R² is Cl; (R³)ₘ is 2-NO₂, 4-F; and n is 0. |
| 51B | R² is Cl; (R³)ₘ is 2-CN, 6-F; and n is 0. |
| 52B | R² is Cl; (R³)ₘ is 2-CF₃, 6-F; and n is 0. |
| 53B | R² is Cl; (R³)ₘ is 2-CF₃, 4-MeO; and n is 0. |
| 54B | R² is Cl; (R³)ₘ is 2-CHF₂O, 6-F; and n is 0. |
| 55B | R² is Cl; (R³)ₘ is 4-MeO; and n is 0. |
| 56B | R² is Cl; (R³)ₘ is 4-EtO; and n is 0. |
| 57B | R² is Cl; (R³)ₘ is 4-MeS; and n is 0. |
| 58B | R² is Cl; (R³)ₘ is 2-CF₃; and n is 0. |
| 59B | R² is Cl; (R³)ₘ is 3-MeC(═O); and n is 0. |
| 60B | R² is Cl; (R³)ₘ is 4-MeOC(═O); and n is 0. |
| 61B | R² is Cl; (R³)ₘ is 4-MeNHC(═O); and n is 0. |
| 62B | R² is Cl; (R³)ₘ is 3-Me₂NC(═O); and n is 0. |
| 63B | R² is Cl; (R³)ₘ is 2,3,6-tri-F; and (R⁵)ₙ is 2-Cl. |
| 64B | R² is Cl; (R³)ₘ is 2,4,6-tri-F; and (R⁵)ₙ is 2-Cl. |
| 65B | R² is Cl; (R³)ₘ is 2,6-di-F, 3-Cl; and (R⁵)ₙ is 2-Cl. |
| 66B | R² is Cl; (R³)ₘ is 2,6-di-F, 4-Me; and (R⁵)ₙ is 2-Cl. |
| 67B | R² is Cl; (R³)ₘ is 2,6-di-F, 4-MeO; and (R⁵)ₙ is 2-Cl. |
| 68B | R² is Cl; (R³)ₘ is 2,6-di-Cl, 4-F; and (R⁵)ₙ is 2-Cl. |
| 69B | R² is Cl; (R³)ₘ is 2,6-di-F; and (R⁵)ₙ is 2-Cl. |
| 70B | R² is Cl; (R³)ₘ is 4-MeO; and (R⁵)ₙ is 2-Cl. |

TABLE 3

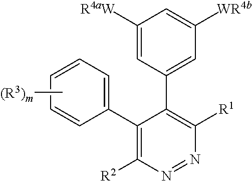

| R¹ | R² | (R₃)ₘ | W | R^{4a} | R^{4b} |
|---|---|---|---|---|---|
| Cl | H | 2,4,6-tri-F | O | Me | Me |
| Me | H | 2,4,6-tri-F | O | Me | Me |
| MeC(═CH₂) | Me | 2,4,6-tri-F | O | Me | Me |
| MeC(═CH₂) | Cl | 2,4,6-tri-F | O | Me | Me |
| MeC(═CH₂) | Me | 2,3,6-tri-F | O | Me | Me |
| MeC(═CH₂) | Cl | 2,3,6-tri-F | O | Me | Me |
| Cl | Me | 2,3,6-tri-F | O | Et | Et |
| Br | Me | 2,3,6-tri-F | O | Et | Et |
| Cl | Me | 2,6-di-F | O | i-Pr | i-Pr |
| Cl | Me | 4-MeO | O | c-Pr | c-Pr |
| Cl | Me | 2,4,6-tri-F | O | Me | Et |
| Me | Cl | 2,4,6-tri-F | O | CF₃ | CF₃ |
| Br | Me | 2,4,6-tri-F | O | CF₃ | CF₃ |
| Cl | Me | 2,4,6-tri-F | S | Me | Me |
| Cl | Me | 4-MeO | S | Me | Me |
| MeC(═CH₂) | Cl | 4-MeO | S | Me | Me |

### Formulation/Utility

A compound of Formula 1 of this invention (including N-oxides and salts thereof) will generally be used as a fungicidal active ingredient in a composition, i.e. formulation, with at least one additional component selected from the group consisting of surfactants, solid diluents and liquid diluents, which serve as a carrier. The formulation or composition ingredients are selected to be consistent with the physical properties of the active ingredient, mode of application and environmental factors such as soil type, moisture and temperature.

Useful formulations include both liquid and solid compositions. Liquid compositions include solutions (including emulsifiable concentrates), suspensions, emulsions (including microemulsions and/or suspoemulsions) and the like, which optionally can be thickened into gels. The general types of aqueous liquid compositions are soluble concentrate, suspension concentrate, capsule suspension, concentrated emulsion, microemulsion and suspo-emulsion. The general types of nonaqueous liquid compositions are emulsifiable concentrate, microemulsifiable concentrate, dispersible concentrate and oil dispersion.

The general types of solid compositions are dusts, powders, granules, pellets, prills, pastilles, tablets, filled films (including seed coatings) and the like, which can be water-dispersible (“wettable”) or water-soluble. Films and coatings formed from film-forming solutions or flowable suspensions are particularly useful for seed treatment. Active ingredient can be (micro)encapsulated and further formed into a suspension or solid formulation; alternatively the entire formulation of active ingredient can be encapsulated (or “overcoated”). Encapsulation can control or delay release of the active ingredient. An emulsifiable granule combines the advantages of both an emulsifiable concentrate formulation and a dry granular formulation. High-strength compositions are primarily used as intermediates for further formulation.

Sprayable formulations are typically extended in a suitable medium before spraying. Such liquid and solid formulations are formulated to be readily diluted in the spray medium, usually water. Spray volumes can range from about one to several thousand liters per hectare, but more typically are in the range from about ten to several hundred liters per hectare. Sprayable formulations can be tank mixed with water or another suitable medium for foliar treatment by aerial or ground application, or for application to the growing medium of the plant. Liquid and dry formulations can be metered directly into drip irrigation systems or metered into the furrow during planting. Liquid and solid formulations can be applied onto seeds of crops and other desirable vegetation as seed treatments before planting to protect developing roots and other subterranean plant parts and/or foliage through systemic uptake.

The formulations will typically contain effective amounts of active ingredient, diluent and surfactant within the following approximate ranges which add up to 100 percent by weight.

| | Weight Percent | | |
|---|---|---|---|
| | Active | | |
| | Ingredient | Diluent | Surfactant |
| Water-Dispersible and Water- | 0.001-90 | 0-99.999 | 0-15 |
| soluble Granules, Tablets and | | | |
| Powders | | | |
| Oil Dispersions, Suspensions, | 1-50 | 40-99 | 0-50 |
| Emulsions, Solutions | | | |
| (including Emulsifiable | | | |
| Concentrates) | | | |
| Dusts | 1-25 | 70-99 | 0-5 |
| Granules and Pellets | 0.001-95 | 5-99.999 | 0-15 |
| High Strength Compositions | 90-99 | 0-10 | 0-2 |

Solid diluents include, for example, clays such as bentonite, montmorillonite, attapulgite, kaolin, gypsum, cellulose, titanium dioxide, zinc oxide, starch, dextrin, sugars (e.g., lactose, sucrose), silica, talc, mica, diatomaceous earth, urea, calcium carbonate, sodium carbonate and bicarbonate, and sodium sulfate. Typical solid diluents are described in Watkins et al., Handbook of Insecticide Dust Diluents and Carriers, 2nd Ed., Dorland Books, Caldwell, N.J.

Liquid diluents include, for example, water, N,N-dimethylalkanamides (e.g., N,N-dimethylformamide), limonene, dimethyl sulfoxide, N-alkylpyrrolidones (e.g., N-methylpyrrolidinone), ethylene glycol, triethylene glycol, propylene glycol, dipropylene glycol, polypropylene glycol, propylene carbonate, butylene carbonate, paraffins (e.g., white mineral oils, normal paraffins, isoparaffins), alkylbenzenes, alkylnaphthalenes, glycerine, glycerol triacetate, sorbitol, aromatic hydrocarbons, dearomatized aliphatics, alkylbenzenes, alkylnaphthalenes, ketones such as cyclohexanone, 2-heptanone, isophorone and 4-hydroxy-4-methyl-2-pentanone, acetates such as isoamyl acetate, hexyl acetate, heptyl acetate, octyl acetate, nonyl acetate, tridecyl acetate and isobornyl acetate, other esters such as alkylated lactate esters, dibasic esters and γ-butyrolactone, and alcohols, which can be linear, branched, saturated or unsaturated, such as methanol, ethanol, n-propanol, isopropyl alcohol, n-butanol, isobutyl alcohol, n-hexanol, 2-ethylhexanol, n-octanol, decanol, isodecyl alcohol, isooctadecanol, cetyl alcohol, lauryl alcohol, tridecyl alcohol, oleyl alcohol, cyclohexanol, tetrahydrofurfuryl alcohol, diacetone alcohol and benzyl alcohol. Liquid diluents also include glycerol esters of saturated and unsaturated fatty acids (typically C₆-C₂₂), such as plant seed and fruit oils (e.g., oils of olive, castor, linseed, sesame, corn (maize), peanut, sunflower, grapeseed, safflower, cottonseed, soybean, rapeseed, coconut and palm kernel), animal-sourced fats (e.g., beef tallow, pork tallow, lard, cod liver oil, fish oil), and mixtures thereof. Liquid diluents also include alkylated fatty acids (e.g., methylated, ethylated, butylated) wherein the fatty acids may be obtained by hydrolysis of glycerol esters from plant and animal sources, and can be purified by distillation. Typical liquid diluents are described in Marsden, Solvents Guide, 2nd Ed., Interscience, New York, 1950.

The solid and liquid compositions of the present invention often include one or more surfactants. When added to a liquid, surfactants (also known as “surface-active agents”) generally modify, most often reduce, the surface tension of the liquid. Depending on the nature of the hydrophilic and lipophilic groups in a surfactant molecule, surfactants can be useful as wetting agents, dispersants, emulsifiers or defoaming agents.

Surfactants can be classified as nonionic, anionic or cationic. Nonionic surfactants useful for the present compositions include, but are not limited to: alcohol alkoxylates such as alcohol alkoxylates based on natural and synthetic alcohols (which may be branched or linear) and prepared from the alcohols and ethylene oxide, propylene oxide, butylene oxide or mixtures thereof; amine ethoxylates, alkanolamides and ethoxylated alkanolamides; alkoxylated triglycerides such as ethoxylated soybean, castor and rapeseed oils; alkylphenol alkoxylates such as octylphenol ethoxylates, nonylphenol ethoxylates, dinonyl phenol ethoxylates and dodecyl phenol ethoxylates (prepared from the phenols and ethylene oxide, propylene oxide, butylene oxide or mixtures thereof); block polymers prepared from ethylene oxide or propylene oxide and reverse block polymers where the terminal blocks are prepared from propylene oxide; ethoxylated fatty acids; ethoxylated fatty esters and oils; ethoxylated methyl esters; ethoxylated tristyrylphenol (including those prepared from ethylene oxide, propylene oxide, butylene oxide or mixtures thereof); fatty acid esters, glycerol esters, lanolin-based derivatives, polyethoxylate esters such as polyethoxylated sorbitan fatty acid esters, polyethoxylated sorbitol fatty acid esters and polyethoxylated glycerol fatty acid esters; other sorbitan derivatives such as sorbitan esters; polymeric surfactants such as random copolymers, block copolymers, alkyd peg (polyethylene glycol) resins, graft or comb polymers and star polymers; polyethylene glycols (pegs); polyethylene glycol fatty acid esters; silicone-based surfactants; and sugar-derivatives such as sucrose esters, alkyl polyglycosides and alkyl polysaccharides.

Useful anionic surfactants include, but are not limited to: alkylaryl sulfonic acids and their salts; carboxylated alcohol or alkylphenol ethoxylates; diphenyl sulfonate derivatives; lignin and lignin derivatives such as lignosulfonates; maleic or succinic acids or their anhydrides; olefin sulfonates; phosphate esters such as phosphate esters of alcohol alkoxylates, phosphate esters of alkylphenol alkoxylates and phosphate esters of styryl phenol ethoxylates; protein-based surfactants; sarcosine derivatives; styryl phenol ether sulfate; sulfates and sulfonates of oils and fatty acids; sulfates and sulfonates of ethoxylated alkylphenols; sulfates of alcohols; sulfates of ethoxylated alcohols; sulfonates of amines and amides such as N,N-alkyltaurates; sulfonates of benzene, cumene, toluene, xylene, and dodecyl and tridecylbenzenes; sulfonates of condensed naphthalenes; sulfonates of naphthalene and alkyl naphthalene; sulfonates of fractionated petroleum; sulfosuccinamates; and sulfosuccinates and their derivatives such as dialkyl sulfosuccinate salts.

Useful cationic surfactants include, but are not limited to: amides and ethoxylated amides; amines such as N-alkyl propanediamines, tripropylenetriamines and dipropylenetetramines, and ethoxylated amines, ethoxylated diamines and propoxylated amines (prepared from the amines and ethylene oxide, propylene oxide, butylene oxide or mixtures thereof); amine salts such as amine acetates and diamine salts; quaternary ammonium salts such as quaternary salts, ethoxylated quaternary salts and diquaternary salts; and amine oxides such as alkyldimethylamine oxides and bis-(2-hydroxyethyl)alkylamine oxides.

Also useful for the present compositions are mixtures of nonionic and anionic surfactants or mixtures of nonionic and cationic surfactants. Nonionic, anionic and cationic surfactants and their recommended uses are disclosed in a variety of published references including McCutcheon's Emulsifiers and Detergents, annual American and International Editions published by McCutcheon's Division, The Manufacturing Confectioner Publishing Co.; Sisely and Wood, Encyclopedia of Surface Active Agents, Chemical Publ. Co., Inc., New York, 1964; and A. S. Davidson and B. Milwidsky, Synthetic Detergents, Seventh Edition, John Wiley and Sons, New York, 1987.

Compositions of this invention may also contain formulation auxiliaries and additives, known to those skilled in the art as formulation aids (some of which may be considered to also function as solid diluents, liquid diluents or surfactants). Such formulation auxiliaries and additives may control: pH (buffers), foaming during processing (antifoams such polyorganosiloxanes), sedimentation of active ingredients (suspending agents), viscosity (thixotropic thickeners), in-container microbial growth (antimicrobials), product freezing (antifreezes), color (dyes/pigment dispersions), wash-off (film formers or stickers), evaporation (evaporation retardants), and other formulation attributes. Film formers include, for example, polyvinyl acetates, polyvinyl acetate copolymers, polyvinylpyrrolidone-vinyl acetate copolymer, polyvinyl alcohols, polyvinyl alcohol copolymers and waxes. Examples of formulation auxiliaries and additives include those listed in McCutcheon's Volume 2: Functional Materials, annual International and North American editions published by McCutcheon's Division, The Manufacturing Confectioner Publishing Co.; and PCT Publication WO 03/024222.

The compound of Formula 1 and any other active ingredients are typically incorporated into the present compositions by dissolving the active ingredient in a solvent or by grinding in a liquid or dry diluent. Solutions, including emulsifiable concentrates, can be prepared by simply mixing the ingredients. If the solvent of a liquid composition intended for use as an emulsifiable concentrate is water-immiscible, an emulsifier is typically added to emulsify the active-containing solvent upon dilution with water. Active ingredient slurries, with particle diameters of up to 2,000 μm can be wet milled using media mills to obtain particles with average diameters below 3 μm. Aqueous slurries can be made into finished suspension concentrates (see, for example, U.S. Pat. No. 3,060,084) or further processed by spray drying to form water-dispersible granules. Dry formulations usually require dry milling processes, which produce average particle diameters in the 2 to 10 μm range. Dusts and powders can be prepared by blending and usually grinding (such as with a hammer mill or fluid-energy mill). Granules and pellets can be prepared by spraying the active material upon preformed granular carriers or by agglomeration techniques. See Browning, “Agglomeration”, Chemical Engineering, Dec. 4, 1967, pp 147-48, Perry's Chemical Engineer's Handbook, 4th Ed., McGraw-Hill, New York, 1963, pages 8-57 and following, and WO 91/13546. Pellets can be prepared as described in U.S. Pat. No. 4,172,714. Water-dispersible and water-soluble granules can be prepared as taught in U.S. Pat. No. 4,144,050, U.S. Pat. No. 3,920,442 and DE 3,246,493. Tablets can be prepared as taught in U.S. Pat. No. 5,180,587, U.S. Pat. No. 5,232,701 and U.S. Pat. No. 5,208,030. Films can be prepared as taught in GB 2,095,558 and U.S. Pat. No. 3,299,566.

For further information regarding the art of formulation, see T. S. Woods, “The Formulator's Toolbox—Product Forms for Modern Agriculture” in Pesticide Chemistry and Bioscience, The Food-Environment Challenge, T. Brooks and T. R. Roberts, Eds., Proceedings of the 9th International Congress on Pesticide Chemistry, The Royal Society of Chemistry, Cambridge, 1999, pp. 120-133. See also U.S. Pat. No. 3,235,361, Col. 6, line 16 through Col. 7, line 19 and Examples 10-41; U.S. Pat. No. 3,309,192, Col. 5, line 43 through Col. 7, line 62 and Examples 8, 12, 15, 39, 41, 52, 53, 58, 132, 138-140, 162-164, 166, 167 and 169-182; U.S. Pat. No. 2,891,855, Col. 3, line 66 through Col. 5, line 17 and Examples 1-4; Klingman, Weed Control as a Science, John Wiley and Sons, Inc., New York, 1961, pp 81-96; Hance et al., Weed Control Handbook, 8th Ed., Blackwell Scientific Publications, Oxford, 1989; and Developments in formulation technology, PJB Publications, Richmond, UK, 2000.

In the following Examples, all percentages are by weight and all formulations are prepared in conventional ways. Compound numbers refer to compounds in Index Table A. Without further elaboration, it is believed that one skilled in the art using the preceding description can utilize the present invention to its fullest extent. The following Examples are, therefore, to be constructed as merely illustrative, and not limiting of the disclosure in any way whatsoever. Percentages are by weight except where otherwise indicated.

### Example A

| High Strength Concentrate | |
|---|---|
| Compound 8 | 98.5% |
| silica aerogel | 0.5% |
| synthetic amorphous fine silica | 1.0% |

### Example B

| Wettable Powder | |
|---|---|
| Compound 9 | 65.0% |
| dodecylphenol polyethylene glycol ether | 2.0% |
| sodium ligninsulfonate | 4.0% |
| sodium silicoaluminate | 6.0% |
| montmorillonite (calcined) | 23.0% |

### Example C

| | |
|---|---|
| Granule | |
| Compound 10 | 10.0% |
| attapulgite granules (low volatile matter, 0.71/0.30 mm; | 90.0% |
| U.S.S. No. 25-50 sieves) | |

### Example D

| Extruded Pellet | |
|---|---|
| Compound 11 | 25.0% |
| anhydrous sodium sulfate | 10.0% |
| crude calcium ligninsulfonate | 5.0% |
| sodium alkylnaphthalenesulfonate | 1.0% |
| calcium/magnesium bentonite | 59.0% |

### Example E

| Emulsifiable Concentrate | |
|---|---|
| Compound 12 | 10.0% |
| polyoxyethylene sorbitol hexoleate | 20.0% |
| C₆-C₁₀ fatty acid methyl ester | 70.0% |

### Example F

| Microemulsion | |
|---|---|
| Compound 15 | 5.0% |
| polyvinylpyrrolidone-vinyl acetate copolymer | 30.0% |
| alkylpolyglycoside | 30.0% |
| glyceryl monooleate | 15.0% |
| water | 20.0% |

### Example G

| Seed Treatment | |
|---|---|
| Compound 17 | 20.00% |
| polyvinylpyrrolidone-vinyl acetate copolymer | 5.00% |
| montan acid wax | 5.00% |
| calcium ligninsulfonate | 1.00% |
| polyoxyethylene/polyoxypropylene block copolymers | 1.00% |
| stearyl alcohol (POE 20) | 2.00% |
| polyorganosilane | 0.20% |
| colorant red dye | 0.05% |
| water | 65.75% |

Water-soluble and water-dispersible formulations are typically diluted with water to form aqueous compositions before application. Aqueous compositions for direct applications to the plant or portion thereof (e.g., spray tank compositions) typically at least about 1 ppm or more (e.g., from 1 ppm to 100 ppm) of the compound(s) of this invention.

The compounds of this invention are useful as plant disease control agents. The present invention therefore further comprises a method for controlling plant diseases caused by fungal plant pathogens comprising applying to the plant or portion thereof to be protected, or to the plant seed to be protected, an effective amount of a compound of the invention or a fungicidal composition containing said compound. The compounds and/or compositions of this invention provide control of diseases caused by a broad spectrum of fungal plant pathogens in the Basidiomycete, Ascomycete, Oomycete and Deuteromycete classes. They are effective in controlling a broad spectrum of plant diseases, particularly foliar pathogens of ornamental, turf, vegetable, field, cereal, and fruit crops. These pathogens include: Oomycetes, including Phytophthora diseases such as Phytophthora infestans, Phytophthora megasperma, Phytophthora parasitica, Phytophthora cinnamomi and Phytophthora capsici, Pythium diseases such as Pythium aphanidermatum, and diseases in the Peronosporaceae family such as Plasmopara viticola, Peronospora spp. (including Peronospora tabacina and Peronospora parasitica), Pseudoperonospora spp. (including Pseudoperonospora cubensis) and Bremia lactucae; Ascomycetes, including Alternaria diseases such as Alternaria solani and Alternaria brassicae, Guignardia diseases such as Guignardia bidwell, Venturia diseases such as Venturia inaequalis, Septoria diseases such as Septoria nodorum and Septoria tritici, powdery mildew diseases such as Erysiphe spp. (including Erysiphe graminis and Erysiphe polygoni), Uncinula necatur, Sphaerotheca fuligena and Podosphaera leucotricha, Pseudocercosporella herpotrichoides, Botrytis diseases such as Botrytis cinerea, Monilinia fructicola, Sclerotinia diseases such as Sclerotinia sclerotiorum, Magnaporthe grisea, Phomopsis viticola, Helminthosporium diseases such as Helminthosporium tritici repentis, Pyrenophora teres, anthracnose diseases such as Glomerella or Colletotrichum spp. (such as Colletotrichum graminicola and Colletotrichum orbiculare), and Gaeumannomyces graminis; Basidiomycetes, including rust diseases caused by Puccinia spp. (such as Puccinia recondita, Puccinia striiformis, Puccinia hordei, Puccinia graminis and Puccinia arachidis), Hemileia vastatrix and Phakopsora pachyrhizi; other pathogens including Rutstroemia floccosum (also known as Sclerontina homoeocarpa); Rhizoctonia spp. (such as Rhizoctonia solani); Fusarium diseases such as Fusarium roseum, Fusarium graminearum and Fusarium oxysporum; Verticillium dahliae; Sclerotium rolfsii; Rynchosporium secalis; Cercosporidium personatum, Cercospora arachidicola and Cercospora beticola; and other genera and species closely related to these pathogens. In addition to their fungicidal activity, the compositions or combinations also have activity against bacteria such as Erwinia amylovora, Xanthomonas campestris, Pseudomonas syringae, and other related species.

Plant disease control is ordinarily accomplished by applying an effective amount of a compound of this invention either pre- or post-infection, to the portion of the plant to be protected such as the roots, stems, foliage, fruit, seeds, tubers or bulbs, or to the media (soil or sand) in which the plants to be protected are growing. The compounds can also be applied to seeds to protect the seeds and seedlings developing from the seeds. The compounds can also be applied through irrigation water to treat plants.

Rates of application for these compounds (i.e. a fungicidally effective amount) can be influenced by factors such as the plant diseases to be controlled, the plant species to be protected, ambient moisture and temperature and should be determined under actual use conditions. One skilled in the art can easily determine through simple experimentation the fungicidally effective amount necessary for the desired level of plant disease control. Foliage can normally be protected when treated at a rate of from less than about 1 g/ha to about 5,000 g/ha of active ingredient. Seed and seedlings can normally be protected when seed is treated at a rate of from about 0.1 to about 10 g per kilogram of seed.

Compounds of this invention can also be mixed with one or more other biologically active compounds or agents including fungicides, insecticides, nematocides, bactericides, acaricides, herbicides, herbicide safeners, growth regulators such as insect molting inhibitors and rooting stimulants, chemosterilants, semiochemicals, repellents, attractants, pheromones, feeding stimulants, plant nutrients, other biologically active compounds or entomopathogenic bacteria, virus or fungi to form a multi-component pesticide giving an even broader spectrum of agricultural protection. Thus the present invention also pertains to a composition comprising a compound of Formula 1 (in a fungicidally effective amount) and at least one additional biologically active compound or agent (in a biologically effective amount) and can further comprise at least one of a surfactant, a solid diluent or a liquid diluent. The other biologically active compounds or agents can be formulated in compositions comprising at least one of a surfactant, solid or liquid diluent. For mixtures of the present invention, one or more other biologically active compounds or agents can be formulated together with a compound of Formula 1, to form a premix, or one or more other biologically active compounds or agents can be formulated separately from the compound of Formula 1, and the formulations combined together before application (e.g., in a spray tank) or, alternatively, applied in succession.

Of note is a composition which in addition to the compound of Formula 1 include at least one fungicidal compound selected from the group consisting of the classes (1) methyl benzimidazole carbamate (MBC) fungicides; (2) dicarboximide fungicides; (3) demethylation inhibitor (DMI) fungicides; (4) phenylamide fungicides; (5) amine/morpholine fungicides; (6) phospholipid biosynthesis inhibitor fungicides; (7) carboxamide fungicides; (8) hydroxy(2-amino)pyrimidine fungicides; (9) anilinopyrimidine fungicides; (10) N-phenyl carbamate fungicides; (11) quinone outside inhibitor (QoI) fungicides; (12) phenylpyrrole fungicides; (13) quinoline fungicides; (14) lipid peroxidation inhibitor fungicides; (15) melanin biosynthesis inhibitors-reductase (MBI-R) fungicides; (16) melanin biosynthesis inhibitors-dehydratase (MBI-D) fungicides; (17) hydroxyanilide fungicides; (18) squalene-epoxidase inhibitor fungicides; (19) polyoxin fungicides; (20) phenylurea fungicides; (21) quinone inside inhibitor (QiI) fungicides; (22) benzamide fungicides; (23) enopyranuronic acid antibiotic fungicides; (24) hexopyranosyl antibiotic fungicides; (25) glucopyranosyl antibiotic: protein synthesis fungicides; (26) glucopyranosyl antibiotic: trehalase and inositol biosynthesis fungicides; (27) cyanoacetamideoxime fungicides; (28) carbamate fungicides; (29) oxidative phosphorylation uncoupling fungicides; (30) organo tin fungicides; (31) carboxylic acid fungicides; (32) heteroaromatic fungicides; (33) phosphonate fungicides; (34) phthalamic acid fungicides; (35) benzotriazine fungicides; (36) benzene-sulfonamide fungicides; (37) pyridazinone fungicides; (38) thiophene-carboxamide fungicides; (39) pyrimidinamide fungicides; (40) carboxylic acid amide (CAA) fungicides; (41) tetracycline antibiotic fungicides; (42) thiocarbamate fungicides; (43) benzamide fungicides; (44) host plant defense induction fungicides; (45) multi-site contact activity fungicides; (46) fungicides other than classes (1) through (45); and salts of compounds of classes (1) through (46).

Further descriptions of these classes of fungicidal compounds are provided below.

(1) “Methyl benzimidazole carbamate (MBC) fungicides” (Fungicide Resistance Action Committee (FRAC) code 1) inhibit mitosis by binding to β-tubulin during microtubule assembly. Inhibition of microtubule assembly can disrupt cell division, transport within the cell and cell structure. Methyl benzimidazole carbamate fungicides include benzimidazole and thiophanate fungicides. The benzimidazoles include benomyl, carbendazim, fuberidazole and thiabendazole. The thiophanates include thiophanate and thiophanate-methyl.

(2) “Dicarboximide fungicides” (Fungicide Resistance Action Committee (FRAC) code 2) are proposed to inhibit a lipid peroxidation in fungi through interference with NADH cytochrome c reductase. Examples include chlozolinate, iprodione, procymidone and vinclozolin.

(3) “Demethylation inhibitor (DMI) fungicides” (Fungicide Resistance Action Committee (FRAC) code 3) inhibit C14-demethylase, which plays a role in sterol production. Sterols, such as ergosterol, are needed for membrane structure and function, making them essential for the development of functional cell walls. Therefore, exposure to these fungicides results in abnormal growth and eventually death of sensitive fungi. DMI fungicides are divided between several chemical classes: azoles (including triazoles and imidazoles), pyrimidines, piperazines and pyridines. The triazoles include azaconazole, bitertanol, bromuconazole, cyproconazole, difenoconazole, diniconazole (including diniconazole-M), epoxiconazole, fenbuconazole, fluquinconazole, flusilazole, flutriafol, hexaconazole, imibenconazole, ipconazole, metconazole, myclobutanil, penconazole, propiconazole, prothioconazole, simeconazole, tebuconazole, tetraconazole, triadimefon, triadimenol, triticonazole and uniconazole. The imidazoles include clotrimazole, imazalil, oxpoconazole, prochloraz, pefurazoate and triflumizole. The pyrimidines include fenarimol and nuarimol. The piperazines include triforine. The pyridines include pyrifenox. Biochemical investigations have shown that all of the above mentioned fungicides are DMI fungicides as described by K. H. Kuck et al. in Modern Selective Fungicides—Properties, Applications and Mechanisms of Action, H. Lyr (Ed.), Gustav Fischer Verlag: New York, 1995, 205-258.

(4) “Phenylamide fungicides” (Fungicide Resistance Action Committee (FRAC) code 4) are specific inhibitors of RNA polymerase in Oomycete fungi. Sensitive fungi exposed to these fungicides show a reduced capacity to incorporate uridine into rRNA. Growth and development in sensitive fungi is prevented by exposure to this class of fungicide. Phenylamide fungicides include acylalanine, oxazolidinone and butyrolactone fungicides. The acylalanines include benalaxyl, benalaxyl-M, furalaxyl, metalaxyl and metalaxyl-M/mefenoxam. The oxazolidinones include oxadixyl. The butyrolactones include ofurace.

(5) “Amine/morpholine fungicides” (Fungicide Resistance Action Committee (FRAC) code 5) inhibit two target sites within the sterol biosynthetic pathway, Δ⁸→>Δ⁷ isomerase and Δ¹⁴ reductase. Sterols, such as ergosterol, are needed for membrane structure and function, making them essential for the development of functional cell walls. Therefore, exposure to these fungicides results in abnormal growth and eventually death of sensitive fungi. Amine/morpholine fungicides (also known as non-DMI sterol biosynthesis inhibitors) include morpholine, piperidine and spiroketal-amine fungicides. The morpholines include aldimorph, dodemorph, fenpropimorph, tridemorph and trimorphamide. The piperidines include fenpropidin and piperalin. The spiroketal-amines include spiroxamine.

(6) “Phospholipid biosynthesis inhibitor fungicides” (Fungicide Resistance Action Committee (FRAC) code 6) inhibit growth of fungi by affecting phospholipid biosynthesis. Phospholipid biosynthesis fungicides include phosphorothiolate and dithiolane fungicides. The phosphorothiolates include edifenphos, iprobenfos and pyrazophos. The dithiolanes include isoprothiolane.

(7) “Carboxamide fungicides” (Fungicide Resistance Action Committee (FRAC) code 7) inhibit Complex II (succinate dehydrogenase) fungal respiration by disrupting a key enzyme in the Krebs Cycle (TCA cycle) named succinate dehydrogenase. Inhibiting respiration prevents the fungus from making ATP, and thus inhibits growth and reproduction. Carboxamide fungicides include benzamides, furan carboxamides, oxathiin carboxamides, thiazole carboxamides, pyrazole carboxamides and pyridine carboxamides. The benzamides include benodanil, flutolanil and mepronil. The furan carboxamides include fenfuram. The oxathiin carboxamides include carboxin and oxycarboxin. The thiazole carboxamides include thifluzamide. The pyrazole carboxamides include furametpyr, penthiopyrad, bixafen, isopyrazam, N-[2-(1S,2R)-[1,1′-bicyclopropyl]-2-ylphenyl]-3-(difluoromethyl)-1-methyl-1H-pyrazole-4-carboxamide and penflufen (N-[2-(1,3-dimethyl-butyl)phenyl]-5-fluoro-1,3-dimethyl-1H-pyrazole-4-carboxamide). The pyridine carboxamides include boscalid.

(8) “Hydroxy(2-amino)pyrimidine fungicides” (Fungicide Resistance Action Committee (FRAC) code 8) inhibit nucleic acid synthesis by interfering with adenosine deaminase. Examples include bupirimate, dimethirimol and ethirimol.

(9) “Anilinopyrimidine fungicides” (Fungicide Resistance Action Committee (FRAC) code 9) are proposed to inhibit biosynthesis of the amino acid methionine and to disrupt the secretion of hydrolytic enzymes that lyse plant cells during infection. Examples include cyprodinil, mepanipyrim and pyrimethanil.

(10) “N-Phenyl carbamate fungicides” (Fungicide Resistance Action Committee (FRAC) code 10) inhibit mitosis by binding to β-tubulin and disrupting microtubule assembly Inhibition of microtubule assembly can disrupt cell division, transport within the cell and cell structure. Examples include diethofencarb.

(11) “Quinone outside inhibitor (QoI) fungicides” (Fungicide Resistance Action Committee (FRAC) code 11) inhibit Complex III mitochondrial respiration in fungi by affecting ubiquinol oxidase. Oxidation of ubiquinol is blocked at the “quinone outside” (Qₒ) site of the cytochrome bc₁ complex, which is located in the inner mitochondrial membrane of fungi. Inhibiting mitochondrial respiration prevents normal fungal growth and development. Quinone outside inhibitor fungicides (also known as strobilurin fungicides) include methoxyacrylate, methoxycarbamate, oximinoacetate, oximinoacetamide, oxazolidinedione, dihydrodioxazine, imidazolinone and benzylcarbamate fungicides. The methoxyacrylates include azoxystrobin, enestroburin (SYP-Z071), picoxystrobin and pyraoxystrobin (SYP-3343). The methoxycarbamates include pyraclostrobin and pyrametostrobin (SYP-4155). The oximinoacetates include kresoxim-methyl and trifloxystrobin. The oximinoacetamides include dimoxystrobin, metominostrobin, orysastrobin, α-[methoxyimino]-N-methyl-2-[[[1-[3-(trifluoromethyl)phenyl]ethoxy]imino]-methyl]benzeneacetamide and 2-[[[3-(2,6-dichlorophenyl)-1-methyl-2-propen-1-ylidene]-amino]oxy]methyl]-α-(methoxyimino)-N-methylbenzeneacetamide. The oxazolidinediones include famoxadone. The dihydrodioxazines include fluoxastrobin. The imidazolinones include fenamidone. The benzylcarbamates include pyribencarb.

(12) “Phenylpyrrole fungicides” (Fungicide Resistance Action Committee (FRAC) code 12) inhibit a MAP protein kinase associated with osmotic signal transduction in fungi. Fenpiclonil and fludioxonil are examples of this fungicide class.

(13) “Quinoline fungicides” (Fungicide Resistance Action Committee (FRAC) code 13) are proposed to inhibit signal transduction by affecting G-proteins in early cell signaling. They have been shown to interfere with germination and/or appressorium formation in fungi that cause powder mildew diseases. Quinoxyfen and tebufloquin are examples of this class of fungicide.

(14) “Lipid peroxidation inhibitor fungicides” (Fungicide Resistance Action Committee (FRAC) code 14) are proposed to inhibit lipid peroxidation which affects membrane synthesis in fungi. Members of this class, such as etridiazole, may also affect other biological processes such as respiration and melanin biosynthesis. Lipid peroxidation fungicides include aromatic carbon and 1,2,4-thiadiazole fungicides. The aromatic carbon fungicides include biphenyl, chloroneb, dicloran, quintozene, tecnazene and tolclofos-methyl. The 1,2,4-thiadiazole fungicides include etridiazole.

(15) “Melanin biosynthesis inhibitors-reductase (MBI-R) fungicides” (Fungicide Resistance Action Committee (FRAC) code 16.1) inhibit the naphthal reduction step in melanin biosynthesis. Melanin is required for host plant infection by some fungi. Melanin biosynthesis inhibitors-reductase fungicides include isobenzofuranone, pyrroloquinolinone and triazolobenzothiazole fungicides. The isobenzofuranones include fthalide. The pyrroloquinolinones include pyroquilon. The triazolobenzothiazoles include tricyclazole.

(16) “Melanin biosynthesis inhibitors-dehydratase (MBI-D) fungicides” (Fungicide Resistance Action Committee (FRAC) code 16.2) inhibit scytalone dehydratase in melanin biosynthesis. Melanin in required for host plant infection by some fungi. Melanin biosynthesis inhibitors-dehydratase fungicides include cyclopropanecarboxamide, carboxamide and propionamide fungicides. The cyclopropanecarboxamides include carpropamid. The carboxamides include diclocymet. The propionamides include fenoxanil.

(17) “Hydroxyanilide fungicides (Fungicide Resistance Action Committee (FRAC) code 17) inhibit C4-demethylase which plays a role in sterol production. Examples include fenhexamid.

(18) “Squalene-epoxidase inhibitor fungicides” (Fungicide Resistance Action Committee (FRAC) code 18) inhibit squalene-epoxidase in ergosterol biosynthesis pathway. Sterols such as ergosterol are needed for membrane structure and function, making them essential for the development of functional cell walls. Therefore exposure to these fungicides results in abnormal growth and eventually death of sensitive fungi. Squalene-epoxidase inhibitor fungicides include thiocarbamate and allylamine fungicides. The thiocarbamates include pyributicarb. The allylamines include naftifine and terbinafine.

(19) “Polyoxin fungicides” (Fungicide Resistance Action Committee (FRAC) code 19) inhibit chitin synthase. Examples include polyoxin.

(20) “Phenylurea fungicides” (Fungicide Resistance Action Committee (FRAC) code 20) are proposed to affect cell division. Examples include pencycuron.

(21) “Quinone inside inhibitor (QiI) fungicides” (Fungicide Resistance Action Committee (FRAC) code 21) inhibit Complex III mitochondrial respiration in fungi by affecting ubiquinol reductase. Reduction of ubiquinol is blocked at the “quinone inside” (Qᵢ) site of the cytochrome bc₁ complex, which is located in the inner mitochondrial membrane of fungi. Inhibiting mitochondrial respiration prevents normal fungal growth and development. Quinone inside inhibitor fungicides include cyanoimidazole and sulfamoyltriazole fungicides. The cyanoimidazoles include cyazofamid. The sulfamoyltriazoles include amisulbrom.

(22) “Benzamide fungicides” (Fungicide Resistance Action Committee (FRAC) code 22) inhibit mitosis by binding to β-tubulin and disrupting microtubule assembly. Inhibition of microtubule assembly can disrupt cell division, transport within the cell and cell structure. Examples include zoxamide.

(23) “Enopyranuronic acid antibiotic fungicides” (Fungicide Resistance Action Committee (FRAC) code 23) inhibit growth of fungi by affecting protein biosynthesis. Examples include blasticidin-S.

(24) “Hexopyranosyl antibiotic fungicides” (Fungicide Resistance Action Committee (FRAC) code 24) inhibit growth of fungi by affecting protein biosynthesis. Examples include kasugamycin.

(25) “Glucopyranosyl antibiotic: protein synthesis fungicides” (Fungicide Resistance Action Committee (FRAC) code 25) inhibit growth of fungi by affecting protein biosynthesis. Examples include streptomycin.

(26) “Glucopyranosyl antibiotic: trehalase and inositol biosynthesis fungicides” (Fungicide Resistance Action Committee (FRAC) code 26) inhibit trehalase in inositol biosynthesis pathway. Examples include validamycin.

(27) “Cyanoacetamideoxime fungicides (Fungicide Resistance Action Committee (FRAC) code 27) include cymoxanil.

(28) “Carbamate fungicides” (Fungicide Resistance Action Committee (FRAC) code 28) are considered multi-site inhibitors of fungal growth. They are proposed to interfere with the synthesis of fatty acids in cell membranes, which then disrupts cell membrane permeability. Propamacarb, propamacarb-hydrochloride, iodocarb, and prothiocarb are examples of this fungicide class.

(29) “Oxidative phosphorylation uncoupling fungicides” (Fungicide Resistance Action Committee (FRAC) code 29) inhibit fungal respiration by uncoupling oxidative phosphorylation. Inhibiting respiration prevents normal fungal growth and development. This class includes 2,6-dinitroanilines such as fluazinam, pyrimidonehydrazones such as ferimzone and dinitrophenyl crotonates such as dinocap, meptyldinocap and binapacryl.

(30) “Organo tin fungicides” (Fungicide Resistance Action Committee (FRAC) code 30) inhibit adenosine triphosphate (ATP) synthase in oxidative phosphorylation pathway. Examples include fentin acetate, fentin chloride and fentin hydroxide.

(31) “Carboxylic acid fungicides” (Fungicide Resistance Action Committee (FRAC) code 31) inhibit growth of fungi by affecting deoxyribonucleic acid (DNA) topoisomerase type II (gyrase). Examples include oxolinic acid.

(32) “Heteroaromatic fungicides” (Fungicide Resistance Action Committee (FRAC) code 32) are proposed to affect DNA/ribonucleic acid (RNA) synthesis. Heteroaromatic fungicides include isoxazole and isothiazolone fungicides. The isoxazoles include hymexazole and the isothiazolones include octhilinone.

(33) “Phosphonate fungicides” (Fungicide Resistance Action Committee (FRAC) code 33) include phosphorous acid and its various salts, including fosetyl-aluminum.

(34) “Phthalamic acid fungicides” (Fungicide Resistance Action Committee (FRAC) code 34) include teclofthalam.

(35) “Benzotriazine fungicides” (Fungicide Resistance Action Committee (FRAC) code 35) include triazoxide.

(36) “Benzene-sulfonamide fungicides” (Fungicide Resistance Action Committee (FRAC) code 36) include flusulfamide.

(37) “Pyridazinone fungicides” (Fungicide Resistance Action Committee (FRAC) code 37) include diclomezine.

(38) “Thiophene-carboxamide fungicides” (Fungicide Resistance Action Committee (FRAC) code 38) are proposed to affect ATP production. Examples include silthiofam.

(39) “Pyrimidinamide fungicides” (Fungicide Resistance Action Committee (FRAC) code 39) inhibit growth of fungi by affecting phospholipid biosynthesis and include diflumetorim.

(40) “Carboxylic acid amide (CAA) fungicides” (Fungicide Resistance Action Committee (FRAC) code 40) are proposed to inhibit phospholipid biosynthesis and cell wall deposition Inhibition of these processes prevents growth and leads to death of the target fungus. Carboxylic acid amide fungicides include cinnamic acid amide, valinamide carbamate and mandelic acid amide fungicides. The cinnamic acid amides include dimethomorph and flumorph. The valinamide carbamates include benthiavalicarb, benthiavalicarb-isopropyl, iprovalicarb, valifenalate and valiphenal. The mandelic acid amides include mandipropamid, N-[2-[4-[[3-(4-chlorophenyl)-2-propyn-1-yl]oxy]-3-methoxyphenyl]ethyl]-3-methyl-2-[(methylsulfonyl)amino]butanamide and N-[2-[4-[[3-(4-chlorophenyl)-2-propyn-1-yl]oxy]-3-methoxyphenyl]ethyl]-3-methyl-2-[(ethylsulfonyl)amino]butanamide.

(41) “Tetracycline antibiotic fungicides” (Fungicide Resistance Action Committee (FRAC) code 41) inhibit growth of fungi by affecting complex 1 nicotinamide adenine dinucleotide (NADH) oxidoreductase. Examples include oxytetracycline.

(42) “Thiocarbamate fungicides (b42)” (Fungicide Resistance Action Committee (FRAC) code 42) include methasulfocarb.

(43) “Benzamide fungicides” (Fungicide Resistance Action Committee (FRAC) code 43) inhibit growth of fungi by delocalization of spectrin-like proteins. Examples include acylpicolide fungicides such as fluopicolide and fluopyram.

(44) “Host plant defense induction fungicides” (Fungicide Resistance Action Committee (FRAC) code P) induce host plant defense mechanisms. Host plant defense induction fungicides include benzo-thiadiazole, benzisothiazole and thiadiazole-carboxamide fungicides. The benzo-thiadiazoles include acibenzolar-5-methyl. The benzisothiazoles include probenazole. The thiadiazole-carboxamides include tiadinil and isotianil.

(45) “Multi-site contact fungicides” inhibit fungal growth through multiple sites of action and have contact/preventive activity. This class of fungicides includes: (45.1) “copper fungicides” (Fungicide Resistance Action Committee (FRAC) code M1)”, (45.2) “sulfur fungicides” (Fungicide Resistance Action Committee (FRAC) code M2), (45.3) “dithiocarbamate fungicides” (Fungicide Resistance Action Committee (FRAC) code M3), (45.4) “phthalimide fungicides” (Fungicide Resistance Action Committee (FRAC) code M4), (45.5) “chloronitrile fungicides” (Fungicide Resistance Action Committee (FRAC) code M5), (45.6) “sulfamide fungicides” (Fungicide Resistance Action Committee (FRAC) code M6), (45.7) “guanidine fungicides” (Fungicide Resistance Action Committee (FRAC) code M7), (45.8) “triazine fungicides” (Fungicide Resistance Action Committee (FRAC) code M8) and (45.9) “quinone fungicides” (Fungicide Resistance Action Committee (FRAC) code M9). “Copper fungicides” are inorganic compounds containing copper, typically in the copper(II) oxidation state; examples include copper oxychloride, copper sulfate and copper hydroxide, including compositions such as Bordeaux mixture (tribasic copper sulfate). “Sulfur fungicides” are inorganic chemicals containing rings or chains of sulfur atoms; examples include elemental sulfur. “Dithiocarbamate fungicides” contain a dithiocarbamate molecular moiety; examples include mancozeb, metiram, propineb, ferbam, maneb, thiram, zineb and ziram. “Phthalimide fungicides” contain a phthalimide molecular moiety; examples include folpet, captan and captafol. “Chloronitrile fungicides” contain an aromatic ring substituted with chloro and cyano; examples include chlorothalonil. “Sulfamide fungicides” include dichlofluanid and tolyfluanid. “Guanidine fungicides” include dodine, guazatine, iminoctadine albesilate and iminoctadine triacetate. “Triazine fungicides” include anilazine. “Quinone fungicides” include dithianon.

(46) “Fungicides other than fungicides of classes (1) through (45)” include certain fungicides whose mode of action may be unknown. These include: (46.1) “thiazole carboxamide fungicides” (Fungicide Resistance Action Committee (FRAC) code U5), (46.2) “phenyl-acetamide fungicides” (Fungicide Resistance Action Committee (FRAC) code U6), (46.3) “quinazolinone fungicides” (Fungicide Resistance Action Committee (FRAC) code U7), (46.4) “benzophenone fungicides” (Fungicide Resistance Action Committee (FRAC) code U8) and (46.5) “triazolopyrimidine fungicides”. The thiazole carboxamides include ethaboxam. The phenyl-acetamides include cyflufenamid and N-[[(cyclopropylmethoxy)amino][6-(difluoromethoxy)-2,3-difluorophenyl]-methylene]benzeneacetamide. The quinazolinones include proquinazid and 2-butoxy-6-iodo-3-propyl-4H-1-benzopyran-4-one. The benzophenones include metrafenone. The (b46) class also includes bethoxazin, neo-asozin (ferric methanearsonate), pyrrolnitrin, quinomethionate, N-[2-[4-[[3-(4-chlorophenyl)-2-propyn-1-yl]oxy]-3-methoxyphenyl]ethyl]-3-methyl-2-[(methylsulfonyl)amino]butanamide, N-[2-[4-[[3-(4-chlorophenyl)-2-propyn-1-yl]oxy]-3-methoxyphenyl]ethyl]-3-methyl-2-[(ethylsulfonyl)amino]butanamide, 2-[[2-fluoro-5-(trifluoromethyl)phenyl]thio]-2-[3-(2-methoxyphenyl)-2-thiazolidinyl-idene]acetonitrile, 3-[5-(4-chlorophenyl)-2,3-dimethyl-3-isoxazolidinyl]pyridine, 4-fluoro-phenyl N-[1-[[[1-(4-cyanophenyl)ethyl]sulfonyl]methyl]propyl]carbamate, 5-chloro-6-(2,4,6-trifluorophenyl)-7-(4-methylpiperidin-1-yl)[1,2,4]triazolo[1,5-a]pyrimidine, N-(4-chloro-2-nitrophenyl)-N-ethyl-4-methylbenzenesulfonamide, N-[[(cyclopropylmethoxy)-amino][6-(difluoromethoxy)-2,3-difluorophenyl]methylene]benzeneacetamide, N-[4-[4-chloro-3-(trifluoromethyl)phenoxy]-2,5-dimethylphenyl]-N-ethyl-N-methylmethanimid-amide and 1-[(2-propenylthio)carbonyl]-2-(1-methylethyl)-4-(2-methylphenyl)-5-amino-1H-pyrazol-3-one. The triazolopyrimidines include ametoctradin.

Therefore of note is a mixture (i.e. composition) comprising a compound of Formula 1 and at least one fungicidal compound selected from the group consisting of the aforedescribed classes (1) through (46). Also of note is a composition comprising said mixture (in fungicidally effective amount) and further comprising at least one additional component selected from the group consisting of surfactants, solid diluents and liquid diluents. Of particular note is a mixture (i.e. composition) comprising a compound of Formula 1 and at least one fungicidal compound selected from the group of specific compounds listed above in connection with classes (1) through (46). Also of particular note is a composition comprising said mixture (in fungicidally effective amount) and further comprising at least one additional surfactant selected from the group consisting of surfactants, solid diluents and liquid diluents.

Examples of other biologically active compounds or agents with which compounds of this invention can be formulated are: insecticides such as abamectin, acephate, acetamiprid, acrinathrin, amidoflumet (S-1955), avermectin, azadirachtin, azinphos-methyl, bifenthrin, bifenazate, buprofezin, carbofuran, cartap, chlorantraniliprole, chlorfenapyr, chlorfluazuron, chlorpyrifos, chlorpyrifos-methyl, chromafenozide, clothianidin, cyantraniliprole (3-bromo-1-(3-chloro-2-pyridinyl)-N-[4-cyano-2-methyl-6-[(methylamino)carbonyl]phenyl]-1H-pyrazole-5-carboxamide), cyflumetofen, cyfluthrin, beta-cyfluthrin, cyhalothrin, lambda-cyhalothrin, cypermethrin, cyromazine, deltamethrin, diafenthiuron, diazinon, dieldrin, diflubenzuron, dimefluthrin, dimethoate, dinotefuran, diofenolan, emamectin, endosulfan, esfenvalerate, ethiprole, fenothiocarb, fenoxycarb, fenpropathrin, fenvalerate, fipronil, flonicamid, flubendiamide, flucythrinate, tau-fluvalinate, flufenerim (UR-50701), flufenoxuron, fonophos, halofenozide, hexaflumuron, hydramethylnon, imidacloprid, indoxacarb, isofenphos, lufenuron, malathion, metaflumizone, metaldehyde, methamidophos, methidathion, methomyl, methoprene, methoxychlor, metofluthrin, milbemycin oxime, monocrotophos, methoxyfenozide, nicotine, nitenpyram, nithiazine, novaluron, noviflumuron (XDE-007), oxamyl, parathion, parathion-methyl, permethrin, phorate, phosalone, phosmet, phosphamidon, pirimicarb, profenofos, profluthrin, pymetrozine, pyrafluprole, pyrethrin, pyridalyl, pyrifluquinazon, pyriprole, pyriproxyfen, rotenone, ryanodine, spinetoram, spinosad, spirodiclofen, spiromesifen (BSN 2060), spirotetramat, sulprofos, tebufenozide, teflubenzuron, tefluthrin, terbufos, tetrachlorvinphos, thiacloprid, thiamethoxam, thiodicarb, thiosultap-sodium, tolfenpyrad, tralomethrin, triazamate, trichlorfon and triflumuron; and biological agents including entomopathogenic bacteria, such as Bacillus thuringiensis subsp. aizawai, Bacillus thuringiensis subsp. kurstaki, and the encapsulated delta-endotoxins of Bacillus thuringiensis (e.g., Cellcap, MPV, MPVII); entomopathogenic fungi, such as green muscardine fungus; and entomopathogenic virus including baculovirus, nucleopolyhedro virus (NPV) such as HzNPV, AfNPV; and granulosis virus (GV) such as CpGV.

Compounds of this invention and compositions thereof can be applied to plants genetically transformed to express proteins toxic to invertebrate pests (such as Bacillus thuringiensis delta-endotoxins). The effect of the exogenously applied fungicidal compounds of this invention may be synergistic with the expressed toxin proteins.

General references for agricultural protectants (i.e. insecticides, fungicides, nematocides, acaricides, herbicides and biological agents) include The Pesticide Manual, 13th Edition, C. D. S. Tomlin, Ed., British Crop Protection Council, Farnham, Surrey, U.K., 2003 and The BioPesticide Manual, 2nd Edition, L. G. Copping, Ed., British Crop Protection Council, Farnham, Surrey, U.K., 2001.

For embodiments where one or more of these various mixing partners are used, the weight ratio of these various mixing partners (in total) to the compound of Formula 1 is typically between about 1:3000 and about 3000:1. Of note are weight ratios between about 1:300 and about 300:1 (for example ratios between about 1:30 and about 30:1). One skilled in the art can easily determine through simple experimentation the biologically effective amounts of active ingredients necessary for the desired spectrum of biological activity. It will be evident that including these additional components may expand the spectrum of diseases controlled beyond the spectrum controlled by the compound of Formula 1 alone.

In certain instances, combinations of a compound of this invention with other biologically active (particularly fungicidal) compounds or agents (i.e. active ingredients) can result in a greater-than-additive (i.e. synergistic) effect. Reducing the quantity of active ingredients released in the environment while ensuring effective pest control is always desirable. When synergism of fungicidal active ingredients occurs at application rates giving agronomically satisfactory levels of fungal control, such combinations can be advantageous for reducing crop production cost and decreasing environmental load.

Of note is a combination of a compound of Formula 1 with at least one other fungicidal active ingredient. Of particular note is such a combination where the other fungicidal active ingredient has different site of action from the compound of Formula 1. In certain instances, a combination with at least one other fungicidal active ingredient having a similar spectrum of control but a different site of action will be particularly advantageous for resistance management. Thus, a composition of the present invention can further comprise a biologically effective amount of at least one additional fungicidal active ingredient having a similar spectrum of control but a different site of action.

Of particular note are compositions which in addition to compound of Formula 1 include at least one compound selected from the group consisting of (1) alkylenebis(dithiocarbamate) fungicides; (2) cymoxanil; (3) phenylamide fungicides; (4) pyrimidinone fungicides; (5) chlorothalonil; (6) carboxamides acting at complex II of the fungal mitochondrial respiratory electron transfer site; (7) quinoxyfen; (8) metrafenone; (9) cyflufenamid; (10) cyprodinil; (11) copper compounds; (12) phthalimide fungicides; (13) fosetyl-aluminum; (14) benzimidazole fungicides; (15) cyazofamid; (16) fluazinam; (17) iprovalicarb; (18) propamocarb; (19) validomycin; (20) dichlorophenyl dicarboximide fungicides; (21) zoxamide; (22) fluopicolide; (23) mandipropamid; (24) carboxylic acid amides acting on phospholipid biosynthesis and cell wall deposition; (25) dimethomorph; (26) non-DMI sterol biosynthesis inhibitors; (27) inhibitors of demethylase in sterol biosynthesis; (28) bc₁ complex fungicides; and salts of compounds of (1) through (28).

Further descriptions of classes of fungicidal compounds are provided below.

Pyrimidinone fungicides (group (4)) include compounds of Formula A1

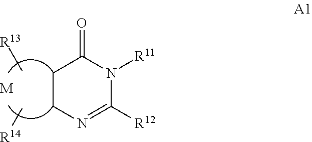

wherein M forms a fused phenyl, thiophene or pyridine ring; R¹¹ is C₁-C₆ alkyl; R¹² is C₁-C₆ alkyl or C₁-C₆ alkoxy; R¹³ is halogen; and R¹⁴ is hydrogen or halogen.

Pyrimidinone fungicides are described in PCT Patent Application Publication WO 94/26722 and U.S. Pat. Nos. 6,066,638, 6,245,770, 6,262,058 and 6,277,858. Of note are pyrimidinone fungicides selected from the group: 6-bromo-3-propyl-2-propyloxy-4(3H)-quinazolinone, 6,8-diiodo-3-propyl-2-propyloxy-4(3H)-quinazolinone, 6-iodo-3-propyl-2-propyloxy-4(3H)-quinazolinone (proquinazid), 6-chloro-2-propoxy-3-propyl-thieno[2,3-d]pyrimidin-4(3H)-one, 6-bromo-2-propoxy-3-propylthieno pyrimidin-4(3H)-one, 7-bromo-2-propoxy-3-propylthieno[3,2-d]pyrimidin-4(3H)-one, 6-bromo-2-propoxy-3-propylpyrido[2,3-d]pyrimidin-4(3H)-one, 6,7-dibromo-2-propoxy-3-propyl-thieno[3,2-d]pyrimidin-4(3H)-one, and 3-(cyclopropylmethyl)-6-iodo-2-(propylthio)pyrido-[2,3-d]pyrimidin-4(3H)-one.

Sterol biosynthesis inhibitors (group (27)) control fungi by inhibiting enzymes in the sterol biosynthesis pathway. Demethylase-inhibiting fungicides have a common site of action within the fungal sterol biosynthesis pathway, involving inhibition of demethylation at position 14 of lanosterol or 24-methylene dihydrolanosterol, which are precursors to sterols in fungi. Compounds acting at this site are often referred to as demethylase inhibitors, DMI fungicides, or DMIs. The demethylase enzyme is sometimes referred to by other names in the biochemical literature, including cytochrome P-450 (14DM). The demethylase enzyme is described in, for example, J. Biol. Chem. 1992, 267, 13175-79 and references cited therein. DMI fungicides are divided between several chemical classes: azoles (including triazoles and imidazoles), pyrimidines, piperazines and pyridines. The triazoles include azaconazole, bromuconazole, cyproconazole, difenoconazole, diniconazole (including diniconazole-M), epoxiconazole, etaconazole, fenbuconazole, fluquinconazole, flusilazole, flutriafol, hexaconazole, imibenconazole, ipconazole, metconazole, myclobutanil, penconazole, propiconazole, prothioconazole, quinconazole, simeconazole, tebuconazole, tetraconazole, triadimefon, triadimenol, triticonazole and uniconazole. The imidazoles include clotrimazole, econazole, imazalil, isoconazole, miconazole, oxpoconazole, prochloraz and triflumizole. The pyrimidines include fenarimol, nuarimol and triarimol. The piperazines include triforine. The pyridines include buthiobate and pyrifenox. Biochemical investigations have shown that all of the above mentioned fungicides are DMI fungicides as described by K. H. Kuck et al. in Modern Selective Fungicides—Properties, Applications and Mechanisms of Action, H. Lyr (Ed.), Gustav Fischer Verlag: New York, 1995, 205-258.

bc₁ Complex Fungicides (group 28) have a fungicidal mode of action which inhibits the bc₁ complex in the mitochondrial respiration chain. The bc₁ complex is sometimes referred to by other names in the biochemical literature, including complex III of the electron transfer chain, and ubihydroquinone:cytochrome c oxidoreductase. This complex is uniquely identified by Enzyme Commission number EC1.10.2.2. The bc₁ complex is described in, for example, J. Biol. Chem. 1989, 264, 14543-48; Methods Enzymol. 1986, 126, 253-71; and references cited therein. Strobilurin fungicides such as azoxystrobin, dimoxystrobin, enestroburin (SYP-Z071), fluoxastrobin, kresoxim-methyl, metominostrobin, orysastrobin, picoxystrobin, pyraclostrobin, pyrametostrobin, pyraoxystrobin and trifloxystrobin are known to have this mode of action (H. Sauter et al., Angew. Chem. Int. Ed. 1999, 38, 1328-1349). Other fungicidal compounds that inhibit the bc₁ complex in the mitochondrial respiration chain include famoxadone and fenamidone.

Alkylenebis(dithiocarbamate)s (group (1)) include compounds such as mancozeb, maneb, propineb and zineb. Phenylamides (group (3)) include compounds such as metalaxyl, benalaxyl, furalaxyl and oxadixyl. Carboxamides (group (6)) include compounds such as boscalid, carboxin, fenfuram, flutolanil, furametpyr, mepronil, oxycarboxin, thifluzamide, penthiopyrad and N-[2-(1,3-dimethylbutyl)phenyl]-5-fluoro-1,3-dimethyl-1H-pyrazole-4-carboxamide (PCT Patent Publication WO 2003/010149), and are known to inhibit mitochondrial function by disrupting complex II (succinate dehydrogenase) in the respiratory electron transport chain. Copper compounds (group (11)) include compounds such as copper oxychloride, copper sulfate and copper hydroxide, including compositions such as Bordeaux mixture (tribasic copper sulfate). Phthalimides (group (12)) include compounds such as folpet and captan. Benzimidazole fungicides (group (14)) include benomyl and carbendazim. Dichlorophenyl dicarboximide fungicides (group (20)) include chlozolinate, dichlozoline, iprodione, isovaledione, myclozolin, procymidone and vinclozolin.

Non-DMI sterol biosynthesis inhibitors (group (26)) include morpholine and piperidine fungicides. The morpholines and piperidines are sterol biosynthesis inhibitors that have been shown to inhibit steps in the sterol biosynthesis pathway at a point later than the inhibitions achieved by the DMI sterol biosynthesis (group (27)). The morpholines include aldimorph, dodemorph, fenpropimorph, tridemorph and trimorphamide. The piperidines include fenpropidin.

Of further note are combinations of compounds of Formula 1 with azoxystrobin, kresoxim-methyl, trifloxystrobin, pyraclostrobin, picoxystrobin, dimoxystrobin, metominostrobin/fenominostrobin, carbendazim, chlorothalonil, quinoxyfen, metrafenone, cyflufenamid, fenpropidine, fenpropimorph, bromuconazole, cyproconazole, difenoconazole, epoxiconazole, fenbuconazole, flusilazole, hexaconazole, ipconazole, metconazole, penconazole, propiconazole, proquinazid, prothioconazole, tebuconazole, triticonazole, famoxadone, prochloraz, penthiopyrad and boscalid (nicobifen).

Preferred for better control of plant diseases caused by fungal plant pathogens (e.g., lower use rate or broader spectrum of plant pathogens controlled) or resistance management are mixtures of a compound of this invention with a fungicide selected from the group: azoxystrobin, kresoxim-methyl, trifloxystrobin, pyraclostrobin, picoxystrobin, dimoxystrobin, metominostrobin/fenominostrobin, quinoxyfen, metrafenone, cyflufenamid, fenpropidine, fenpropimorph, cyproconazole, epoxiconazole, flusilazole, metconazole, propiconazole, proquinazid, prothioconazole, tebuconazole, triticonazole, famoxadone and penthiopyrad. Specifically preferred mixtures (compound numbers refer to compounds in Index Table A) are selected from the group: combinations of Compound 8, Compound 9, Compound 10, Compound 11, Compound 12, Compound 15, Compound 17 with azoxystrobin, combinations of Compound 8, Compound 9, Compound 10, Compound 11, Compound 12, Compound 15, Compound 17 with kresoxim-methyl, combinations of Compound 8, Compound 9, Compound 10, Compound 11, Compound 12, Compound 15, Compound 17 with trifloxystrobin, combinations of Compound 8, Compound 9, Compound 10, Compound 11, Compound 12, Compound 15, Compound 17 with picoxystrobin, combinations of Compound 8, Compound 9, Compound 10, Compound 11, Compound 12, Compound 15, Compound 17 with metominostrobin/fenominostrobin, combinations of Compound 8, Compound 9, Compound 10, Compound 11, Compound 12, Compound 15, Compound 17 with quinoxyfen, combinations of Compound 8, Compound 9, Compound 10, Compound 11, Compound 12, Compound 15, Compound 17 with metrafenone, combinations of Compound 8, Compound 9, Compound 10, Compound 11, Compound 12, Compound 15, Compound 17 with fenpropidine, combinations of Compound 8, Compound 9, Compound 10, Compound 11, Compound 12, Compound 15, Compound 17 with fenpropimorph, combinations of Compound 8, Compound 9, Compound 10, Compound 11, Compound 12, Compound 15, Compound 17 with cyproconazole, combinations of Compound 8, Compound 9, Compound 10, Compound 11, Compound 12, Compound 15, Compound 17 with epoxiconazole, combinations of Compound 8, Compound 9, Compound 10, Compound 11, Compound 12, Compound 15, Compound 17 with flusilazole, combinations of Compound 8, Compound 9, Compound 10, Compound 11, Compound 12, Compound 15, Compound 17 with metconazole, combinations of Compound 8, Compound 9, Compound 10, Compound 11, Compound 12, Compound 15, Compound 17 with propiconazole, combinations of Compound 8, Compound 9, Compound 10, Compound 11, Compound 12, Compound 15, Compound 17 with proquinazid, combinations of Compound 8, Compound 9, Compound 10, Compound 11, Compound 12, Compound 15, Compound 17 with prothioconazole, combinations of Compound 8, Compound 9, Compound 10, Compound 11, Compound 12, Compound 15, Compound 17 with tebuconazole, combinations of Compound 8, Compound 9, Compound 10, Compound 11, Compound 12, Compound 15, Compound 17 with triticonazole, combinations of Compound 8, Compound 9, Compound 10, Compound 11, Compound 12, Compound 15, Compound 17 with famoxadone, Compound 8, Compound 9, Compound 10, Compound 11, Compound 12, Compound 15, Compound 17 with penthiopyrad, combinations of Compound 8, Compound 9, Compound 10, Compound 11, Compound 12, Compound 15, Compound 17 with 3-(difluoromethyl)-1-methyl-N-(3′,4′,5′-trifluoro[1,1′-biphenyl]-2-yl)-1H-pyrazole-4-carboxamide, combinations of Compound 8, Compound 9, Compound 10, Compound 11, Compound 12, Compound 15, Compound 17 with 5-ethyl-6-octyl-[1,2,4]triazole[1,5-a]pyrimidin-7-amine, and Compound 8, Compound 9, Compound 10, Compound 11, Compound 12, Compound 15, Compound 17 with Initium®.

The Tests shown below in Table A, Biological Examples of The Invention demonstrate the control efficacy of compounds of this invention on specific pathogens. The pathogen control protection afforded by the compounds is not limited, however, to these species. See Index Table A for compound descriptions. The following abbreviations are used in Index Table A: Me is methyl, i-Pr is isopropyl, MeO is methoxy and CN is cyano. The abbreviation “Ex.” stands for “Example” and is followed by a number indicating in which example the compound is prepared. A dash (“-”) in the column “(R⁵)ₘ” of Index Table A indicates m is 0 and hydrogen is present at all available positions.

INDEX TABLE A

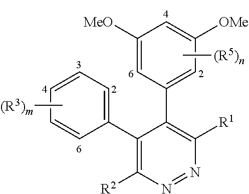

| | | | | | m.p. |
|---|---|---|---|---|---|
| Compound | R¹ | R² | (R³)ₘ | (R⁵)ₘ | (° C.) |
| 1 | Cl | Me | 4-Cl | — | * |
| 2 | Cl | Me | 4-MeO | — | * |
| 3 | Cl | Me | 2,4-di-F | — | * |
| 4 (Ex. 6) | Cl | Me | 2,6-di-F | — | ** |
| 5 (Ex. 3) | H | Cl | 2,4,6-tri-F | — | ** |
| 6 | H | Me | 2,4,6-tri-F | — | * |
| 7 (Ex. 5) | H | CH₂Cl | 2,4,6-tri-F | — | ** |
| 8 (Ex. 1) | Me | Cl | 2,4,6-tri-F | — | ** |
| 9 (Ex. 2) | Me | Me | 2,4,6-tri-F | — | ** |
| 10 | Me | Cl | 2,6-di-F, | — | * |
| | | | 4-MeO | | |
| 11 | Me | Me | 2,6-di-F, | — | * |
| | | | 4-MeO | | |
| 12 (Ex. 5) | Cl | Me | 2,4,6-tri-F | — | ** |
| 13 | Cl | Me | 4-F | — | 177-178 |
| 14 | MeC(═O) | Me | 4-MeO | — | * |
| 15 | Me | Cl | 2,3,6-tri-F | — | * |
| 16 | i-Pr | Cl | 2,4,6-tri-F | — | * |
| 17 | Me | Me | 2,3,6-tri-F | — | * |
| 18 | i-Pr | Me | 2,4,6-tri-F | — | * |
| 19 | Me₂C(OH) | Me | 4-MeO | — | * |
| 20 (Ex. 8) | MeC(═O) | Me | 2,4,6-tri-F | — | ** |
| 21 (Ex. 9) | Me₂C(OH) | Me | 2,4,6-tri-F | — | ** |
| 22 (Ex. 4) | H | Me | 2,4,6-tri-F | — | ** |
| [note 1] | | | | | |
| 23 (Ex. 7) | Me | Cl | 2,4,6-tri-F | 2-Cl | ** |
| 24 | Me | Cl | 2,6-di-F | — | * |
| 25 [note 1] | Me | Cl | 2,6-di-F | — | * |
| 26 (Ex. 10) | CH₂Cl | Cl | 2,6-di-F | — | ** |
| 27 (Ex. 11) | CH₂CN | Cl | 2,6-di-F | — | ** |
| 28 | Me | Cl | 2,6-di-F | 2-Cl | 190-192 |
| 29 [note 1] | Me | Cl | 2,6-di-F | 2-Cl | * |
| 30 | CH₂Cl | Cl | 2,6-di-F | 2-Cl | * |
| 31 | Me | Me | 2,6-di-F | — | 170-172 |
| 32 | Me | Cl | 2,4-di-F | — | 196-199 |
| 33 | Me | F | 2,6-di-F | — | 150-151 |
| 34 | Me | Me | 2,6-di-F | 2,6- | 189-191 |
| | | | | di-Cl | |
| 35 | CH₂CN | Me | 2,6-di-F | — | * |
| 36 | Me | F | 2,6-di-F | 2-Cl | 187-189 |
| 38 | Me | Cl | 2-Cl, 6-F | — | 171-172 |
| 39 | Me | Me | 2-Cl, 6-F | — | 144-146 |
| 40 | Me | Me | 2,4-di-F | — | 178-180 |
| 41 | Me | Cl | 2-Cl, 6-F | 2-Cl | 198-200 |
| 42 | Me | Cl | 2-Cl, 6-F | 2,6- | 235-237 |
| | | | | di-Cl | |
| 43 | Me | Me | 2-Cl, 6-F | 2-Cl | 132-134 |
| 44 | Me | Cl | 2,4-di-F | 2-Cl | 166-167 |
| 45 | Me | Cl | 2,4-di-F | 4-Cl | 130-131 |
| 46 | Me | F | 2,4-di-F | — | 179-181 |
| 47 | Me | F | 2-Cl, 6-F | — | 137-139 |
| 48 | Me | Cl | 2-Cl, 4-F | — | 221-223 |
| 49 | Me | Me | 2,4-di-F | 2-Cl | 160-161 |
| 50 | Me | Me | 2,4-di-F | 4-Cl | 158-160 |
| 51 | Me | F | 2,4-di-F | 2-Cl | 172-173 |
| 52 | Me | Me | 2-Cl, 4-F | — | 195-196 |
| 53 | Me | Cl | 2-Cl, 4-F | 2-Cl | 156-157 |

[Note 1]:
Mixture of 1-, and 2-N-oxide.
*See Index Table B for ¹H NMR data.
**See synthesis example for ¹H NMR data.

TABLE B
INDEX

| Compd. | |
|---|---|
| No. | ¹H NMR Data (CDCl₃ solution unless indicated otherwise)^{a} |
| 1 | δ 7.4 (d, 2H), 7.27-7.2 (m, 5H), 7.0 (d, 2H), 6.19 (s, 1H), |
| | 5.9 (d, 2H), 3.49 (s, 6H), 2.57 (s, 3H). |
| 2 | δ 6.92 (d, 2H), 6.81 (d, 2H), 6.3 (s, 1H), 6.17 (d, 2H), 3.78 |
| | (s, 3H), 3.67 (s, 6H). |
| 3 | δ 6.9 (m, 1H), 6.8 (m, 2H), 6.36 (s, 1H), 6.3 (s, 1H), 6.1 (s, |
| | 1H), 3.60 (s, 6H ), 2.50 (s, 3H). |
| 6 | δ 9.1 (s, 1H), 6.7 (t, 2H), 6.42 (s, 1H), 6.28 (s, 2H), 3.7 (s, |
| | 6H), 2.58 (s, 3H). |
| 10 | δ 6.40-6.37 (s and d, 3H), 6.21 (s, 2H), 3.76 (s, 3H), 3.71 (s, |
| | 6H), 2.55 (s, 3H). |
| 11 | δ 6.40-6.37 (m, 3H), 6.21 (s, 2H), 3.76 (s, 3H), 3.71 (s, 6H), |
| | 2.53 (s, 3H), 2.49 (s, 3H). |
| 14 | δ 6.9 (d, 2H), 6.8 (d, 2H), 6.3 (s, 1H), 6.0 (d, 2H), 3.78 (s, |
| | 3H), 3.62 (s, 6H), 2.69 (s, 3H), 2.59 (s, 3H). |
| 15 | δ 7.1 (m, 1H), 6.8 (m, 1H), 6.3 (s, 1H), 6.2 (s, 2H), 3.71 (s, |
| | 6H), 2.58 (s, 3H). |
| 16 | δ 6.6 (t, 2H), 6.3 (s, 1H), 6.19 (d, 2H), 3.71 (s, 6H), 3.0 (m, |
| | 1H), 1.35 (d, 6H). |
| 17 | δ 7.1 (m, 1H), 6.8 (m, 1H), 6.34 (s, 1H), 6.2 (d, 2H), 3.70 (s, |
| | 6H), 2.56 (s, 3H), 2.51 (s, 3H). |
| 18 | δ 6.6 (t, 2H), 6.35 (s, 1H), 6.18 (d, 2H), 3.70 (s, 6H), 3.0 (m, |
| | 1H), 2.49 (s, 3H), 1.34 (d, 6H). |
| 19 | δ 6.8 (d, 2H), 6.7 (d, 2H), 6.3 (s, 1H), 6.13 (d, 2H), 6.0 (br s, |
| | 1H) 3.76 (s, 3H), 3.67 (s, 6H), 2.48 (s, 3H), 1.41 (s, 6H). |
| 24 | δ 7.3 (m, 1H), 6.85 (t, 2H), 6.35 (s, 1H), 6.2 (s, 2H), 3.69 (s, |
| | 6H), 2.57 (s, 3H). |
| 25 | δ 7.3 (m, 1H), 6.84 (t, 2H), 6.35 (s, 1H), 6.21 (d, 2H), 3.69 |
| | (s, 6H), 2.34 (s, 3H). |
| 29 | δ 7.3 (m, 1H), 6.8 (m, 2H), 6.4 (s, 1H), 6.2 (s, 1H), 3.83 (s, |
| | 3H), 3.69 (s, 3H), 2.29 (s, 3H). |
| 30 | δ 7.3 (m, 1H), 6.8 (t, 2H), 6.4 (s, 1H), 6.38 (s, 1H), 4.86 (d |
| | of d, 1H), 4.56 (d of d, 1H), 3.83 (s, 3H), 3.7 (s, 3H). |
| 35 | δ 7.3 (m, 1H), 6.8 (m, 2H), 6.4 (s, 1H), 6.2 (s, 2H), 3.92 (s, |
| | 2H), 3.6 (s, 6H), 2.56 (s, 3H). |

^{a1}H NMR data are in ppm downfield from tetramethylsilane. Couplings are designated by (s)—singlet, (d)—doublet, (d of d)—doublet of doublets, (t)—triplet, (m)—multiplet, (br s)—broad singlet.

### Biological Examples of the Invention

General protocol for preparing test suspensions for Tests A-F: the test compounds were first dissolved in acetone in an amount equal to 3% of the final volume and then suspended at the desired concentration (in ppm) in acetone and purified water (50/50 mix) containing 250 ppm of the surfactant Trem® 014 (polyhydric alcohol esters). The resulting test suspensions were then used in Tests A-F. Spraying a 200 ppm test suspension to the point of run-off on the test plants was the equivalent of a rate of 500 g/ha. An asterisk “*” next to the rating value indicates a 40 ppm test suspension.

### Test A

The test suspension was sprayed to the point of run-off on tomato seedlings. The following day the seedlings were inoculated with a spore suspension of Botrytis cinerea (the causal agent of tomato Botrytis) and incubated in saturated atmosphere at 20° C. for 48 h, and then moved to a growth chamber at 24° C. for 3 days, after which time visual disease ratings were made.

### Test B

The test suspension was sprayed to the point of run-off on tomato seedlings. The following day the seedlings were inoculated with a spore suspension of Alternaria solani (the causal agent of tomato early blight) and incubated in a saturated atmosphere at 27° C. for 48 h, and then moved to a growth chamber at 20° C. for 5 days, after which time visual disease ratings were made.

### Test C

The test suspension was sprayed to the point of run-off on wheat seedlings. The following day the seedlings were inoculated with a spore suspension of Septoria nodorum (the causal agent of wheat glume blotch) and incubated in a saturated atmosphere at 24° C. for 48 h, and then moved to a growth chamber at 20° C. for 6 days, after which time visual disease ratings were made.

### Test D

The test suspension was sprayed to the point of run-off on wheat seedlings. The following day the seedlings were inoculated with a spore suspension of Septoria tritici (the causal agent of wheat leaf blotch) and incubated in saturated atmosphere at 24° C. for 48 h, and then moved to a growth chamber at 20° C. for 19 days, after which time visual disease ratings were made.

### Test E1

Wheat seedlings were inoculated with a spore suspension of Puccinia recondita f. sp. tritici (the causal agent of wheat leaf rust) and incubated in a saturated atmosphere at 20° C. for 24 h, and then moved to a growth chamber at 20° C. for 2 days. At the end of this time the test suspension was sprayed to the point of run-off on the wheat seedlings, and then the seedlings were moved to a growth chamber at 20° C. for 6 days, after which time visual disease ratings were made.

### Test E2

The test suspension was sprayed to the point of run-off on wheat seedlings. The following day the seedlings were inoculated with a spore suspension of Puccinia recondita f. sp. tritici (the causal agent of wheat leaf rust) and incubated in a saturated atmosphere at 20° C. for 24 h, and then moved to a growth chamber at 20° C. for 7 days, after which time visual disease ratings were made.

### Test F

The test suspension was sprayed to the point of run-off on wheat seedlings. The following day the seedlings were inoculated with a spore dust of Erysiphe graminis, (the causal agent of wheat powdery mildew) and incubated in a growth chamber at 20° C. for 8 days, after which time visual disease ratings were made.

Results for Tests A-F are given in Table A. In the Table, a rating of 100 indicates 100% disease control and a rating of 0 indicates no disease control (relative to the controls). A dash (-) indicates no test results. All results are for 200 ppm except where followed by “*”, which indicates 40 ppm.

TABLE A

| Cmpd | Test | Test | Test | Test | Test | Test | Test |
|---|---|---|---|---|---|---|---|
| No. | A | B | C | D | E1 | E2 | F |
| 1 | 0 | 0 | 0 | 0 | — | 0 | 0 |
| 2 | 95 | 58 | 0 | 62 | — | 74 | 26 |
| 3 | 91 | 31 | 0 | 97 | — | 97 | 79 |
| 4 | 95 | 97 | 0 | 98 | — | 100 | 95 |
| 5 | 99 | 100 | 100 | 98 | 99 | 100 | 100 |
| 6 | 97 | 100 | 96 | 93 | — | 100 | 100 |
| 7 | 91 | 26 | 94 | 96 | — | 98 | 99 |
| 8 | 100* | 100* | 100* | 99* | 100* | 100* | 99* |
| 9 | 100* | 99* | 100* | 100* | 100* | 100* | 100* |
| 10 | 100* | 100* | 98* | 99* | 100* | 100* | 99* |
| 11 | 100* | 100* | 99* | 100* | 100* | 100* | 100* |
| 12 | 99* | 100* | 99* | 99* | 100* | 100* | 100* |
| 13 | 8 | 0 | 0 | 45 | — | 19 | 39 |
| 14 | 99 | 0 | 0 | 99 | 16 | 95 | 84 |
| 15 | 100 | 100 | 84 | 99 | 100 | 100 | 99 |
| 16 | 99 | 99 | 92 | 99 | 100 | 100 | 100 |
| 17 | 100 | 100 | 99 | 99 | 100 | 100 | 100 |
| 18 | 100 | 99 | 82 | 99 | 100 | 100 | 100 |
| 19 | 84* | 99* | 0* | 97* | 0* | 96* | 27* |
| 20 | 99* | 26* | 0* | 96* | 41* | 99* | 98* |
| 21 | 100* | 99* | 40* | 98* | 100* | 100* | 100* |
| 22 | — | — | — | — | — | — | — |
| 23 | 100* | 100* | 100* | 100* | 100* | 100* | 100* |
| 24 | 100* | 26* | 55* | 100* | 26* | 100* | 98* |
| 25 | 100* | 9* | 0* | 100* | 9* | 100* | 95* |
| 26 | 99* | 0* | 0* | 99* | 0* | 97* | 0* |
| 27 | 100 | 99 | 100 | 100 | 0 | 100 | 98 |
| 28 | 100 | 100 | 78 | 100 | — | 100 | 100 |
| 29 | — | — | — | — | — | — | — |
| 30 | — | — | — | — | — | — | — |
| 31 | 100 | 99 | 100 | 100 | — | 99 | 98 |
| 32 | 99 | 99 | 78 | 99 | 0 | 99 | 89 |
| 33 | 99 | 100 | 99 | 100 | 60 | 100 | 99 |
| 34 | 100 | 0 | 0 | 100 | — | 100 | 97 |
| 35 | 98 | 99 | 99 | 100 | 46 | 100 | 99 |
| 36 | 99 | 100 | 60 | 100 | 100 | 100 | 100 |
| 38 | 100 | 99 | 99 | 100 | 85 | 100 | 100 |
| 39 | 100 | 99 | 99 | 100 | 99 | 100 | 98 |
| 40 | 100 | 99 | 97 | 100 | 0 | 100 | 95 |
| 41 | 99 | 99 | 0 | 100 | 86 | 100 | 100 |
| 42 | 0 | 0 | 0 | 60 | 0 | 89 | 42 |
| 43 | 62 | 82 | 99 | 100 | 0 | 100 | 100 |
| 44 | 66 | 99 | 64 | 100 | 96 | 100 | 100 |
| 45 | 100 | 100 | 97 | 100 | 100 | 100 | 100 |
| 46 | 99 | 100 | 84 | 100 | 0 | 98 | 98 |
| 47 | — | 100 | 99 | — | 100 | 99 | 99 |
| 48 | — | 100 | 0 | — | 0 | 99 | 97 |
| 49 | — | 99 | 99 | — | 100 | 100 | 100 |
| 50 | — | 100 | 100 | — | 100 | 100 | 100 |
| 51 | 99 | 100 | 0 | — | 0 | 100 | 99 |
| 52 | 99 | 94 | 87 | — | 9 | 100 | 99 |
| 53 | 99 | 99 | 98 | — | 98 | 100 | 100 |

What is claimed is:

1. A compound selected from Formula 1, N-oxides, and salts thereof, 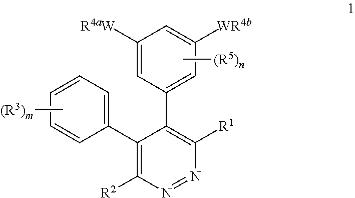
wherein
each W is independently O or S;
R¹ and R² are each independently H, halogen, cyano, C₁-C₆ alkyl, C₂-C₆ alkenyl, C₂-C₆ alkynyl, C₃-C₆ cycloalkyl, C₁-C₆ haloalkyl, C₂-C₆ haloalkenyl, C₁-C₆ hydroxyalkyl, C₂-C₆ cyanoalkyl, C₁-C₆ alkoxy, C₁-C₆ haloalkoxy, C₁-C₆ alkylthio, C₁-C₆ haloalkylthio, C₂-C₆ alkylcarbonyl or C₂-C₆ alkoxycarbonyl;
each R³ is independently halogen, cyano, nitro, C₁-C₆ alkyl, C₂-C₆ alkenyl, C₂-C₆ alkynyl, C₃-C₆ cycloalkyl, C₁-C₆ haloalkyl, C₂-C₆ haloalkenyl, C₁-C₆ alkoxy, C₁-C₆ haloalkoxy, C₁-C₆ alkylthio, C₁-C₆ haloalkylthio, C₂-C₆ alkylcarbonyl, C₂-C₆ alkoxycarbonyl, C₂-C₆ alkylaminocarbonyl, C₃-C₆ dialkylaminocarbonyl or C₃-C₆ trialkylsilyl;
R^{4a} and R^{4b} are each independently C₁-C₄ alkyl, C₁-C₄ haloalkyl or C₃-C₆ cycloalkyl;
each R⁵ is independently halogen, cyano, nitro, C₁-C₄ alkyl, C₁-C₄ haloalkyl, C₁-C₄ alkoxy, C₁-C₄ haloalkoxy, C₁-C₄ alkylthio or C₁-C₄ haloalkylthio;
m is 1, 2, 3, 4 or 5; and
n is 0, 1 or 2;
provided that:
(a) when R¹ is H, chloro, cyano or methoxy, then R² is not the same as R¹; and
(b) the compound is other than 4-(2,6-difluorophenyl)-5-(3,5-dimethoxyphenyl)-3-methyl-6-(1-methylethenyl)pyridazine, 4-(2,4-difluorophenyl)-5-(3,5-dimethoxyphenyl)-3-methyl-6-(1-methylethenyl)pyridazine or 4-(3,5-dimethoxyphenyl)-5-(4-methoxyphenyl)-6-methyl-3-(1-methylethenyl)pyridazine.

2. A compound of claim 1 wherein:
each W is O;
R¹ and R² are each independently H, halogen, C₁-C₄ alkyl, C₂-C₄ alkenyl, C₁-C₄ haloalkyl, C₁-C₄ alkoxy, C₂-C₄ alkylcarbonyl, C₁-C₄ hydroxyalkyl or C₂-C₄ cyanoalkyl;
each R³ is independently halogen, cyano, C₁-C₃ alkyl, C₁-C₃ haloalkyl, C₁-C₃ alkoxy, C₁-C₃ haloalkoxy or C₁-C₃ alkylthio;
R^{4a} and R^{4b} are each methyl;
each R⁵ is independently halogen, cyano, C₁-C₂ alkyl, C₁-C₂ alkoxy or C₁-C₂ haloalkyl;
m is 2 or 3; and
n is 0 or 1.

3. A compound of claim 2 wherein:
R¹ and R² are each independently H, halogen, C₁-C₂ alkyl, C₂ alkenyl, C₁-C₂ alkoxy, C₂ alkylcarbonyl or C₁-C₃ hydroxyalkyl;
each R³ is independently Cl, F, cyano, methyl, methoxy or methylthio; and
each R⁵ is independently Cl, F, methyl or methoxy.

4. A compound of claim 3 wherein:
R¹ and R² are each independently H, Br, Cl, methyl, C₂ alkenyl or methoxy;
each R³ is independently Cl, F, methyl or methoxy; and
n is 0.

5. A compound of claim 4 wherein:
R¹ and R² are each independently Cl or methyl; and
at least one R³ substituent is attached at an ortho position.

6. A compound of claim 5 wherein:
two R³ substituents are attached at the ortho positions and one R³ substituent is attached at a meta or the para position; and
m is 3.

7. A compound of claim 1 which is selected from the group consisting of:
3-chloro-5-(3,5-dimethoxyphenyl)-6-methyl-4-(2,4,6-trifluorophenyl)pyridazine;
4-(3,5-dimethoxyphenyl)-3,6-dimethyl-5-(2,4,6-trifluorophenyl)pyridazine;
3-chloro-4-(2,6-difluoro-4-methoxyphenyl)-5-(3,5-dimethoxyphenyl)-6-methylpyridazine;
4-(2,6-difluoro-4-methoxyphenyl)-5-(3,5-dimethoxyphenyl)-3,6-dimethylpyridazine;
3-chloro-5-(3,5-dimethoxyphenyl)-4-(2,4,6-trifluorophenyl)pyridazine;
5-(3,5-dimethoxyphenyl)-3-methyl-4-(2,4,6-trifluorophenyl)pyridazine;
3-chloro-5-(3,5-dimethoxyphenyl)-6-methyl-4-(2,3,6-trifluorophenyl)pyridazine;
4-(3,5-dimethoxyphenyl)-3,6-dimethyl-5-(2,3,6-trifluorophenyl)pyridazine; and
3-chloro-4-(3,5-dimethoxyphenyl)-6-methyl-5-(2,4,6-trifluorophenyl)pyridazine.

8. A fungicidal composition comprising (a) a compound of claim 1; and (b) at least one other fungicide.

9. A fungicidal composition comprising (a) a compound of claim 1; and (b) at least one additional component selected from the group consisting of surfactants, solid diluents and liquid diluents.

10. A method for controlling plant diseases caused by fungal plant pathogens comprising applying to the plant or portion thereof, or to the plant seed, a fungicidally effective amount of a compound of claim 1.
